(19) United States
(12) Patent Application Publication
(10) Pub. No.: US 2006/0247637 A1
(43) Pub. Date: Nov. 2, 2006

(54) System and method for dynamic skeletal stabilization

(76) Inventors: Dennis Colleran, North Attleboro, MA (US); Carolyn Rogers, Frisco, TX (US); James Spitler, Taunton, MA (US); Scott Schorer, Duxbury, MA (US)

Correspondence Address: CARR LLP (IST), 670 FOUNDERS SQUARE, 900 JACKSON STREET, DALLAS, TX, 75202, US

(21) Appl. No.: 11/443,236

(22) Filed: May 30, 2006

Related U.S. Application Data
(63) Continuation-in-part of application No. 10/914,751, filed on Aug. 9, 2004.
(63) Continuation-in-part of application No. PCT/US05/27996, filed on Aug. 8, 2005.
(60) Provisional application No. 60/637,324, filed on Dec. 16, 2004.
(60) Provisional application No. 60/656,126, filed on Feb. 24, 2005.
(60) Provisional application No. 60/685,705, filed on May 27, 2005.
(60) Provisional application No. 60/685,760, filed on May 27, 2005.
(60) Provisional application No. 60/692,943, filed on Jun. 22, 2005.
(60) Provisional application No. 60/693,300, filed on Jun. 22, 2005.

(51) Int. Cl. A61F 2/30

(52) U.S. Cl. 606/61

(57) ABSTRACT

Provided are embodiments of spine stabilization systems, devices, and methods. In one example, a device includes a brace adapted to span between first and second bone anchors. The brace may include a first member and a second member. In this example, the brace may allow for movement between the first and second members that is restricted to a three dimensional curved path having a substantially constant radius about a center of rotation positioned outside of the brace.

### CROSS-REFERENCED APPLICATIONS

This application is a continuation-in-part, and claims priority from, the following co-pending and commonly assigned patent applications: U.S. patent application Ser. No. 10/914,751, entitled “SYSTEM AND METHOD FOR DYNAMIC SKELETAL STABILIZATION,” filed Aug. 9, 2004; PCT application serial no. PCT/US2005/027996, entitled “SYSTEM AND METHOD FOR DYNAMIC SKELETAL STABILIZATION,” filed on Aug. 8, 2005; and U.S. patent application Ser. No. 11/303,138, entitled “THREE COLUMN SUPPORT DYNAMIC STABILIZATION SYSTEM AND METHOD OF USE,” filed on Dec. 16, 2005. This application also claims priority from the following commonly assigned patent applications: U.S. provisional application Ser. No. 60/637,324, entitled “THREE COLUMN SUPPORT DYNAMIC STABILIZATION SYSTEM AND METHOD OF USE,” filed Dec. 16, 2004; U.S. provisional application Ser. No. 60/656,126, entitled “SYSTEM AND METHOD FOR DYNAMIC STABILIZATION,” filed Feb. 24, 2005; U.S. provisional application Ser. No. 60/685,705, entitled “FOUR-BAR DYNAMIC STABILIZATION DEVICE,” filed May 27, 2005; U.S. provisional application Ser. No. 60/685,760, entitled “SLIDABLE POST DYNAMIC STABILIZATION DEVICE,” filed May 27, 2005; U.S. provisional application Ser. No. 60/692,943, entitled “SPHERICAL MOTION DYNAMIC STABILIZATION DEVICE,” filed Jun. 22, 2005; U.S. provisional application Ser. No. 60/693,300, entitled “SPHERICAL PLATE DYNAMIC STABILIZATION DEVICE,” filed Jun. 22, 2005. The disclosures of all of the above applications are herein incorporated by reference.

### FIELD OF THE INVENTION

This disclosure relates to skeletal stabilization and, more particularly, to systems and method for stabilization of human spines and, even more particularly, to dynamic stabilization techniques.

### BACKGROUND

The human spine is a complex structure designed to achieve a myriad of tasks, many of them of a complex kinematic nature. The spinal vertebrae allow the spine to flex in three axes of movement relative to the portion of the spine in motion. These axes include the horizontal (bending either forward/anterior or aft/posterior), roll (bending to either left or right side) and vertical (twisting of the shoulders relative to the pelvis).

In flexing about the horizontal axis, into flexion (bending forward or anterior) and extension (bending backward or posterior), vertebrae of the spine must rotate about the horizontal axis to various degrees of rotation. The sum of all such movement about the horizontal axis of produces the overall flexion or extension of the spine. For example, the vertebrae that make up the lumbar region of the human spine move through roughly an arc of 15° relative to its adjacent or neighboring vertebrae. Vertebrae of other regions of the human spine (e.g., the thoracic and cervical regions) have different ranges of movement. Thus, if one were to view the posterior edge of a healthy vertebrae, one would observe that the edge moves through an arc of some degree (e.g., of about 15° in flexion and about 5° in extension if in the lumbar region) centered around an elliptical center of rotation. During such rotation, the anterior (front) edges of neighboring vertebrae move closer together, while the posterior edges move farther apart, compressing the anterior of the spine. Similarly, during extension, the posterior edges of neighboring vertebrae move closer together, while the anterior edges move farther apart, compressing the posterior of the spine. Also during flexion and extension, the vertebrae move in horizontal relationship to each other, providing up to 2-3 mm of translation.

In a normal spine, the vertebrae also permit right and left lateral bending. Accordingly, right lateral bending indicates the ability of the spine to bend over to the right by compressing the right portions of the spine and reducing the spacing between the right edges of associated vertebrae. Similarly, left lateral bending indicates the ability of the spine to bend over to the left by compressing the left portions of the spine and reducing the spacing between the left edges of associated vertebrae. The side of the spine opposite that portion compressed is expanded, increasing the spacing between the edges of vertebrae comprising that portion of the spine. For example, the vertebrae that make up the lumbar region of the human spine rotate about an axis of roll, moving through roughly an arc of 10° relative to its neighbor vertebrae, throughout right and left lateral bending.

Rotational movement about a vertical axis relative to the portion of the spine moving is also desirable. For example, rotational movement can be described as the clockwise or counter-clockwise twisting rotation of the vertebrae, such as during a golf swing.

The inter-vertebral spacing (between neighboring vertebrae) in a healthy spine is maintained by a compressible and somewhat elastic disc. The disc serves to allow the spine to move about the various axes of rotation and through the various arcs and movements required for normal mobility. The elasticity of the disc maintains spacing between the vertebrae, allowing room or clearance for compression of neighboring vertebrae during flexion and lateral bending of the spine. In addition, the disc allows relative rotation about the vertical axis of neighboring vertebrae, allowing twisting of the shoulders relative to the hips and pelvis. Clearance between neighboring vertebrae maintained by a healthy disc is also important to allow nerves from the spinal chord to extend out of the spine, between neighboring vertebrae, without being squeezed or impinged by the vertebrae.

In situations (based upon injury or otherwise) where a disc is not functioning properly, the inter-vertebral disc tends to compress or become degenerated. The compressed or degenerated disc may cause pressure to be exerted on nerves extending from the spinal cord by this reduced inter-vertebral spacing. Various other types of nerve problems may be experienced in the spine, such as exiting nerve root compression in the neural foramen, passing nerve root compression, and ennervated annulus (where nerves grow into a cracked/compromised annulus, causing pain every time the disc/annulus is compressed), as examples. Many medical procedures have been devised to alleviate such nerve compression and the pain that results from nerve pressure. Many of these procedures revolve around attempts to prevent the vertebrae from moving too close to each other, thereby maintaining space for the nerves to exit without being impinged upon by movements of the spine.

In one such procedure, screws are embedded in adjacent vertebrae pedicles and rigid rods or plates are then secured between the screws. In such a situation, the pedicle screws (which are in effect extensions of the vertebrae) then press against the rigid spacer which serves to distract the degenerated disc space, maintaining adequate separation between the neighboring vertebrae so as to prevent the vertebrae from compressing the nerves. This prevents nerve pressure due to extension of the spine; however, when the patient then tries to bend forward (putting the spine in flexion), the posterior portions of at least two vertebrae are effectively held together. Furthermore, the lateral bending or rotational movement between the affected vertebrae is significantly reduced due to the rigid connection of the spacers. Overall movement of the spine is reduced as more vertebrae are distracted by such rigid spacers. This type of spacer not only limits the patient's movements, but also places additional stress on other portions of the spine (typically, the stress placed on adjacent vertebrae without spacers being the worse), often leading to further complications at a later date.

In many situations, spinal dynamic stabilization may be preferred to alleviate these problems that relate to the human spine. When inter-vertebral spacing is compromised by a degenerated disc, restoring vertebral movement which allows flexion, extension and/or rotation may be preferred. Additionally, vertebral movement about all three axes may be preferred to emulate a healthy spine.

### SUMMARY

Numerous embodiments and aspects of the present invention are disclosed. For instance, in one embodiment, a spine stabilization device is provided. The device comprises a brace adapted to span between a first bone anchor and a second bone anchor. The brace includes a first joint and a second joint, wherein the brace allows for movement between the first joint and the second joint such that the movement of the second joint with respect to the first joint is generally restricted to vertical and horizontal movement along a three dimensional curved path surface having a substantially constant radius about a center of rotation.

In another embodiment, a spine stabilization system is disclosed. The system comprises a first bone anchor, a second bone anchor, and a brace spanning between the first bone anchor and the second bone anchor. The brace includes a first member coupled to the first bone anchor and a second member coupled to the second bone anchor. The first member and the second member are slideably mated along a portion of their longitudinal lengths such that the movement of the second member with respect to the first member is generally restricted to vertical and horizontal movement along a three dimensional curved path surface having a substantially constant radius about a center of rotation.

In yet another embodiment, a method for spine stabilization is provided. The method comprises inserting a first bone anchor into a first vertebra, inserting a second bone anchor into a second vertebra, attaching a first joint to the first bone anchor, attaching a second joint to the second bone anchor, and interconnecting the first joint and the second joint to create a brace that spans the first bone anchor and the second bone anchor, such that the first joint and the second joint are slideably mated along a portion of their longitudinal lengths. The brace allows for movement between the first joint and the second joint such that the movement of the first joint with respect to the second joint is generally restricted to vertical and horizontal movement along a three dimensional curved path surface having a substantially constant radius about a center of rotation.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the invention is intended to encompass within its scope such processes, machines, manufacture, compositions of matter, means, methods, or steps.

### BRIEF DESCRIPTION OF THE DRAWINGS

For a more complete understanding of the present invention and the advantages thereof, reference is now made to the following Detailed Description taken in conjunction with the accompanying drawings, in which:

FIGS. 1A-1C are side views of one embodiment of a dynamic stabilization device illustrating two dimensional rotation of adjacent vertebrae.

FIG. 1D is an isometric view of a portion of a spine illustrating three axes and three dimensional motion around a center of rotation.

FIGS. 2A-2G illustrate embodiments of dynamic braces that may allow three dimensional movement.

FIGS. 3A-3I illustrate an alternative embodiment of a dynamic brace that may allow three dimensional movement.

FIGS. 4A-4J illustrate an alternative dynamic brace that may allow three dimensional movement.

FIGS. 5A-5J illustrate an alternative embodiment of a dynamic brace that may allow three dimensional movement using a four-bar design.

FIG. 6 is an isometric view illustrating an alternative embodiment of a dynamic brace that may allow three dimensional movement using a four-bar design.

FIGS. 7A-7I illustrate an alternative embodiment of a dynamic brace that may allow three dimensional movement using an optimized four-bar design.

FIG. 8A illustrates an embodiment of a system incorporating several aspects of the present invention.

FIGS. 8B-8F illustrate three dimensional movement of one embodiment of a system incorporating several aspects of the present invention.

FIG. 9 illustrates an alternative embodiment of a dynamic device that may allow two dimensional rotation about an axis using a four-bar design.

FIGS. 10A-10E illustrate an alternative embodiment of a dynamic device that may allow two dimensional rotation about an axis using a slider design.

FIG. 10F illustrates an embodiment of a system incorporating two of the dynamic devices illustrated in FIGS. 10A-10E.

FIG. 10G illustrates an embodiment of a member that may be used within the system of FIG. 10F.

FIGS. 11A-11C illustrate an alternative embodiment of a dynamic device that may allow three dimensional movement using a device which anchors to the spinous processes.

FIG. 12 illustrates an embodiment of a cover that could be used with any of the disclosed embodiments.

### DETAILED DESCRIPTION

In the following discussion, numerous specific details are set forth to provide a thorough understanding of the present invention. However, those skilled in the art will appreciate that the present invention may be practiced without such specific details.

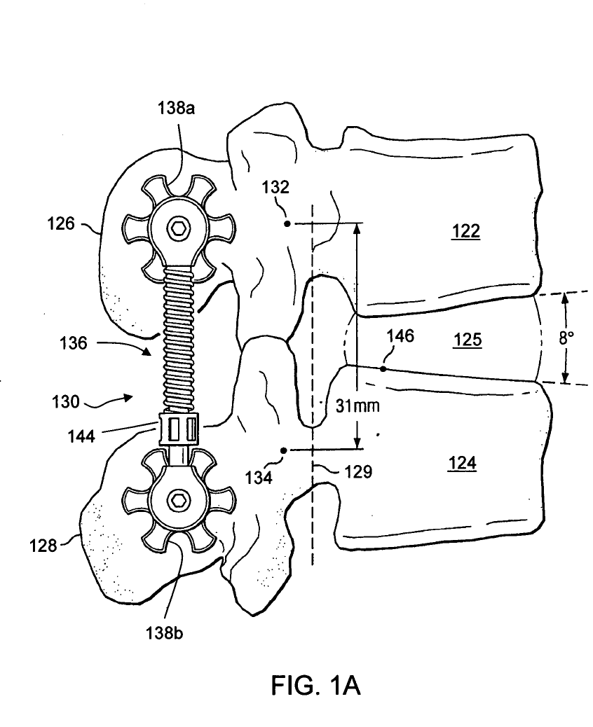

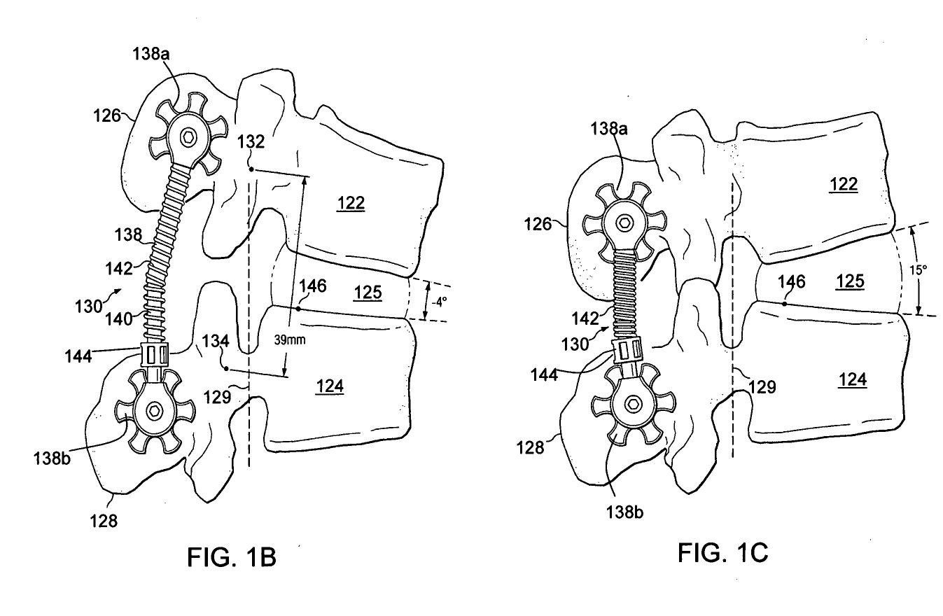

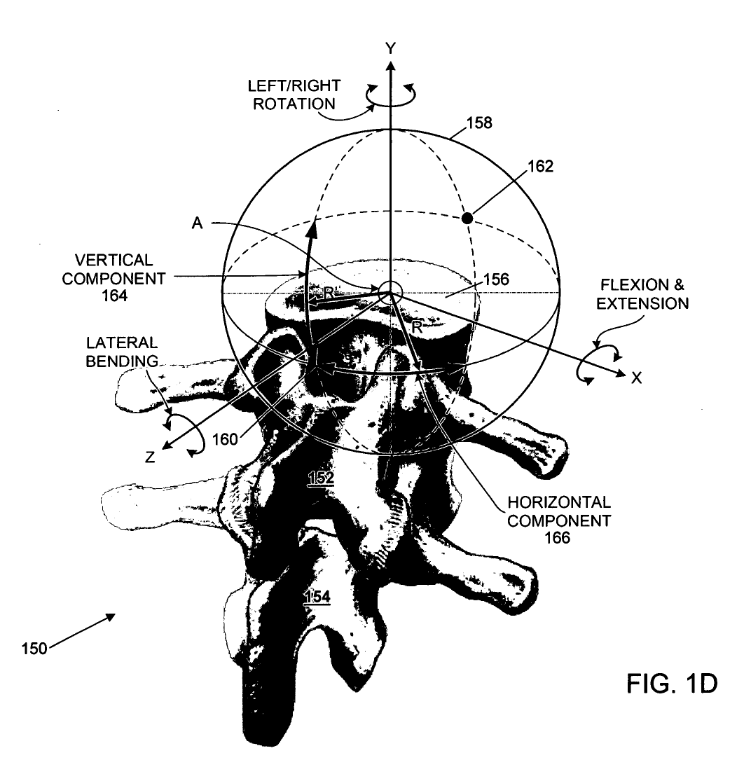

FIGS. 1A-1C show an upper vertebra 122 and a lower vertebra 124 (which could, for example, be L4, L5 or any other vertebrae) separated by disc 125. Also shown are an upper spinous process 126 and a lower spinous process 128. A space or region 129 between the vertebrae 122 and 124 is where nerves would typically emerge from the spinal column. FIG. 1A shows this exemplary portion of a skeletal system in the neutral position. In this position, the angle between planes corresponding to the vertebral end-plates of the adjacent vertebrae could be, for example, 8°.

An exemplary dynamic stabilization device 130 (e.g., a brace or support member) is illustrated coupling the adjacent spinous processes 126 and 128. Typically, a similar device may be anchored to the other side of the spinous processes (not shown). However, in some embodiments, such a dynamic stabilization device may be used unilaterally. It is also noted that, in certain embodiments, the attachment of the dynamic stabilization device 130 to the relative spinous process 126 or 128 should be as anterior on the spinous process as practical. For example, the junction of the lamina and the spinous process may be a strong fixation point. Note that, while not shown, an extension (or another stabilization device) may extend to a next adjacent spinous process if multiple vertebrae are to be stabilized.

It should also be noted that the dynamic stabilization device 130 is just one example of a posterior stabilization device which may be used in accordance with various aspects of the present invention. The use of dynamic stabilization device 130 is for purposes of illustrating the movement of vertebrae. Other posterior stabilization devices may be used. In other aspects of the present invention, a stabilization device could be anchored to the pedicles at, for example, upper pedicle point 132 and lower pedicle point 134.

In this example, the dynamic stabilization device 130 may include a brace 136, which spans between bone anchors 138a and 138b. As shown in FIG. 1B, the brace 136 may include an upper elongated portion 138 and a lower elongated portion 140. The upper elongated portion 138 is free to move with respect to the lower elongated portion 140 along its longitudinal axis in a telescoping manner. This motion may be limited or controlled, in part, by a spring 142. In some embodiments, a stop 144 may allow the spring 142 (or springs) to be effectively lengthened or shortened. This lengthening or shortening may change the range of motion and, in certain embodiments, may change the force the spring exerts. This may, in turn, change the force between the elongated portions 138 and 140.

FIG. 1B shows the dynamic stabilization device 130 with vertebrae 122 and 124 in the flexed position (e.g., when a person is bending forward). Note that in this illustration, the spinous process 126 has moved up and to the right (anterior) as the spine is bent forward (flexion). A typical movement distance for the posterior of the spinous process is patient specific and may be approximately 4-16 mm. In this exemplary embodiment, the spring 142 has expanded along with the brace 136 to allow the spinous process 126 to move upward and forward, rotating about a center of rotation 146. Thus, the vertebra 122 rotates with respect to the vertebra 124 during flexion (e.g., when a person bends forward). In this illustrative example, the rotation point or the center point about which vertebra 124 rotates is illustrated as point 146. In a completely natural movement (without any devices), the rotation point may not be a constant point but may move in an change as the vertebrae move from extension to flexion or from anterior to posterior translation.

When fully in flexion, the front surfaces of vertebrae 122 and 124 form an angle of, for example, −4°, which, in this example, is a change of 12° from the neutral position. On the other hand, if the vertebrae goes into extension by, for example, 3°, the total range of motion may be about 15° as shown in FIG. 1C, having a center of rotation located around point 146. The center of rotation of the spine does not change from flexion to extension or with side bending. However, the “Instantaneous Axis of Rotation” (IAR) changes throughout the rotation arc. The positional sum of all of the IARs may be thought of as the one point that is called the Center of Rotation (COR). When the spine moves through flexion and extension, the motion of the adjacent vertebrae may modeled as an arc having a center which corresponds to the center of rotation. In certain embodiments, a dynamic brace may be adjusted to move the center of rotation forward-backward (X axis) and upward-downward (Y axis), as will be discussed later.

As illustrated in FIG. 1B, the dynamic stabilization device 130 includes the spring 142, which in certain embodiments, may act in compression as a progressive breaking mechanism or an extension limiter to limit the compression applied to nerves extending from the region 129. Note that as between FIGS. 1A and 1B, the respective pedicles have separated by approximately 8 mm. The range shown (31 mm to 39 mm) is but one example. Other patients may have other starting and ending points depending upon their particular physical structure and medical condition. In certain embodiments, the dynamic stabilization device 130 may enable the pedicles (vertebrae) and facets to move through a range of motion which allows them to separate during flexion.

As illustrated in FIG. 1C, the spring 142 may also serve to stabilize the spine when in extension. In both flexion and extension, the limit of movement may be controlled by the limits of upper elongated portion 138 and lower elongated portion 140 along their longitudinal length.

The rotation illustrated in FIGS. 1A-1C includes two dimensional rotation. In other words, due to flexion or extension, the vertebra 122 rotates about a horizontal axis coming out of the plane of FIG. 1A at point 146. While stabilization systems that permit two dimensional movement may represent an improvement over fusion systems, a healthy human spine allows movement in three dimensions.

FIG. 1D illustrates a portion of a spine 150 shown in an isometric view. The spine portion 150 comprises an upper vertebra 152 and a lower vertebra 154. In an actual spine, an intervertebral disc (similar to disc 125 of FIG. 1A) would be located on top of a vertebral plate 156 of the vertebra 152, but is omitted in the present example for clarity. Furthermore, an upper adjacent vertebra (similar to vertebra 125) would be positioned above the intervertebral disc. This upper adjacent vertebra is also omitted for clarity.

In FIG. 1D, imaginary “X”, “Y”, and “Z” axes are superimposed upon the spine portion 150. The intersection of the axes may be defined to be a center of rotation “A” which, for purposes of this discussion, is positioned above the vertebral plate 156 within the intervertebral space. Natural spine motion may be modeled in relation to the X, Y, and Z axes. As previously discussed, flexion or extension movement may be modeled as a rotation of the vertebra about the X-axis. In addition, lateral bending (bending towards the right or left) may be modeled as rotation about the Z-axis. Rotation (twisting the torso in relation to the legs) may be modeled as rotation about the Y-axis. Thus, the relative natural movement of the vertebrae of spine occurs in three dimensions with respect to the three illustrated axes.

Certain aspects of the present invention allow movement along the surface of an imaginary three dimensional curved body, such as a sphere or ellipsoid. For discussion purposes, a sphere 158 is shown superimposed upon spine portion 150. The center of the sphere 158 is at the center of rotation “A.” A posterior stabilization device that allows a point on an upper vertebra (not shown) to move in relation to a corresponding point on the vertebra 152 by following a path that is restricted to the surface of the sphere 158 would allow movement about all three axes. When used with certain aspects of the invention, the term “restricted” refers to a substantially two dimensional curvilinear path or three dimensional curved path wherein the instantaneous axis of rotation (which may change throughout the full range of motion of the brace), may be within an ellipsoid or another region.

For instance, assume a path has a starting point at point 160 which is on the surface of the sphere 158. Further assume that the path has an ending point 162 which is also on the surface of the sphere 158. Thus, it can be seen that the path between point 160 and point 162 that follows the surface of the sphere 158 has a vertical component 164 and a horizontal component 166. Movement that is restricted to the vertical curved component 164 is considered to be two dimensional movement or rotation about the X-axis (as discussed in relation to FIGS. 1A-1C). Movement that is restricted to the horizontal component 166 is also two dimensional movement, but represents rotation about the Y-axis. The combination of the vertical curved component and the horizontal curved component represents three-dimensional movement about the center of rotation “A”.

It is understood that the use of a sphere is for purposes of example and the present invention is not limited to a spherical path. If the path between points is restricted to the surface of a sphere, the path will have a constant radius of curvature “R” with respect to the center of rotation “A.” In certain aspects of the present invention, the horizontal component 166 may have a radius of curvature R and the vertical component 164 may have a radius of curvature R′. Thus, if the radius of curvature R equals that of R′ and they have the same center of rotation, the path would be on a sphere as illustrated. On the other hand, if R′ does not equal R, then the imaginary three dimensional curved body could be an ellipsoid or another three dimensional curved surface. Certain aspects of the present invention also contemplate a curved vertical component 164 and a straight or nearly straight horizontal component.

In certain embodiments, dynamic braces may form a radius between the members of the brace and center of rotation “A” about which the brace is capable of motion in a vertical and/or horizontal direction.

Dynamic Systems and Devices that Permit Three Dimensional Movement:

Several embodiments and aspects of devices and implants that permit freedom of movement between neighboring vertebrae in flexion/extension, lateral bending, and rotation directions, while restraining the degree of movement generally along an imaginary three dimensional curved surface will now be discussed.

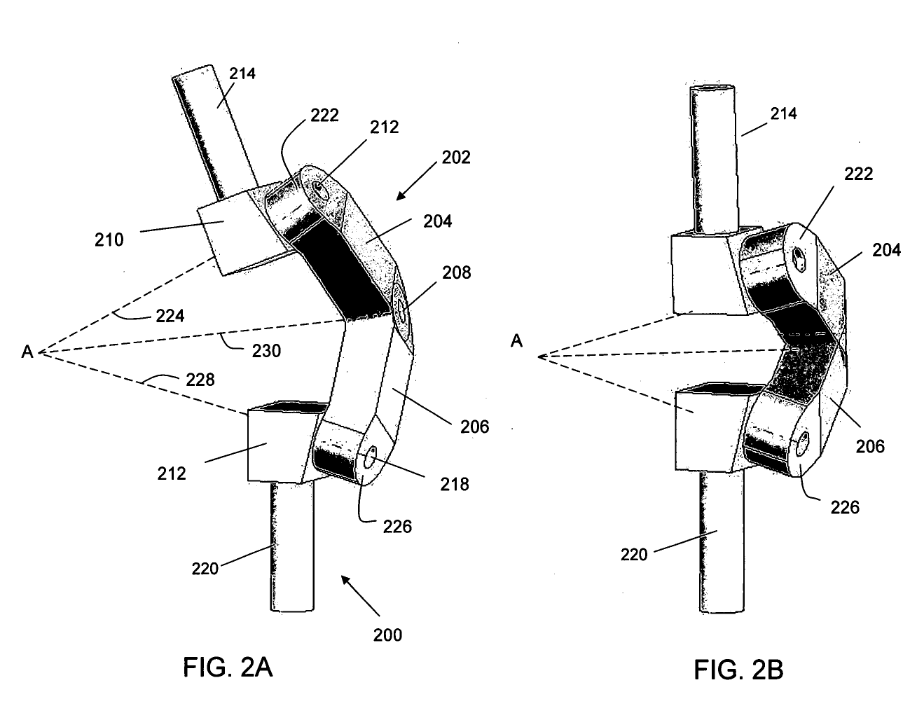

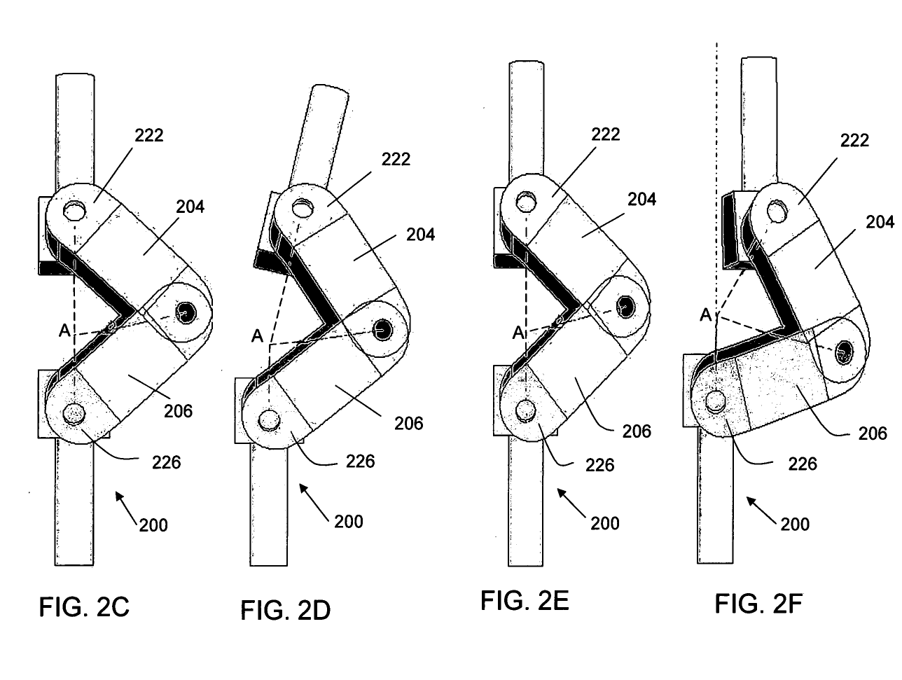

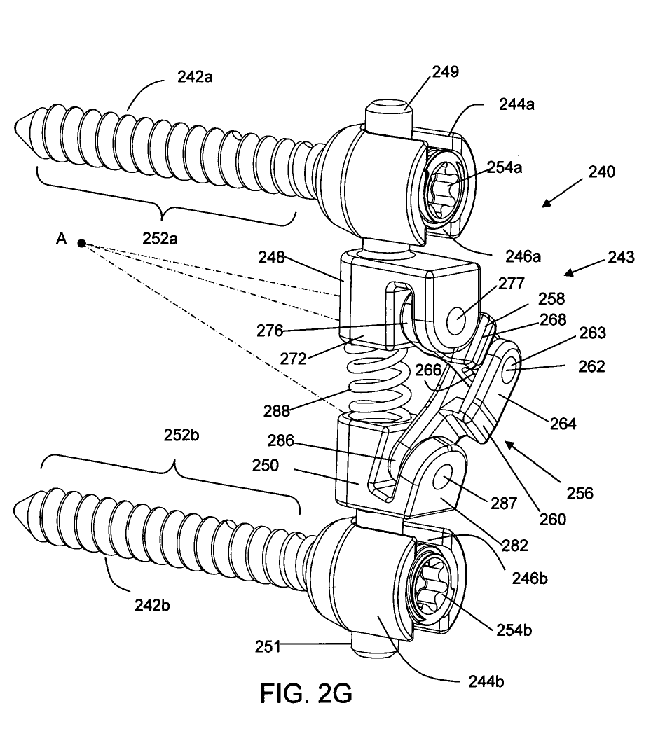

Referring to FIG. 2A, which depicts a conceptual representation of a dynamic stabilization device 200 that may be coupled to two adjacent vertebrae (not shown in FIG. 2A) through the use of attachment techniques that will be discussed later. As will be discussed, the dynamic stabilization device 200 may be coupled to the adjacent pedicles so that they may move with respect to each other by following a curved motion in all three directions around a common center of rotation.

As illustrated in FIG. 2A, the dynamic stabilization device 200 permits freedom of movement between neighboring vertebrae in flexion/extension, lateral bending, and rotation directions, while restraining the degree of movement generally along an imaginary three dimensional curved surface, such as a spherical shell about a spherical center of rotation “A”. In this embodiment, the dynamic stabilization device 200 includes an elbow 202 having an upper spherical strip 204 and a lower spherical strip 206 pivotably interconnected at a pivot connection 208. An outboard end 222 of the upper strip 204 may be pivotably connected to a boss 210 with a pivot connection 212. In this embodiment, the boss 210 may be coupled to an upper shank or connecting member 214. The outboard end 226 of lower strip 206 may be pivotably connected to a lower boss 216 by a pivot 218. The lower boss 216 may be coupled to a lower shank or connecting member 220. As will be explained later, the upper and lower shank members 214 and 220 may each be coupled to a bone anchor (not shown) for connection to a vertebrae, such as vertebrae 122 and 124 of FIG. 1A.

In the illustrative embodiment, the pivot connections 208, 212, and 218 may be hinged connections having a pin (not shown) joining the respective members. Each pin has a longitudinal axis about which the connection members can rotate. In some embodiments, the upper strip 204 and lower strip 206 may be strips of a sphere having its center at point “A.” In yet other embodiments, the strips may be shaped in a way that allows the pivot connections to maintain an axis of rotation that intersects point “A.” For instance, the outboard end 222 of the upper strip 204 may be bent about an axis longitudinal to the strip and about an axis perpendicular to the strip, so that, when the elbow 202 is positioned in its approximately middle position, the axis of pivot 224 points downwardly and inwardly towards point “A.” The outboard end 226 of the lower strip 206 may be similarly bent about an axis longitudinal to the strip and about an axis perpendicular to the strip, so that the axis of pivot 228 points upwardly and inwardly towards point “A.” Interconnected ends of upper strip 204 and of lower strip 206 are each bent about an axis longitudinal to the strip and also perpendicular to each of the respective strips so that the interconnection axis 230 between the strips points inwardly towards the same point “A.”

Because the longitudinal axis of each pin in the pivot connections 208, 212 and 218 of elbow 202 points generally towards the same central point “A”, the elbow 202 only allows movement (or “restricts movement to”) of the pivoted ends of the strips to the space generally occupied by the surface of an imaginary spherical shell having a center of rotation at “A”, as the vertebrae move relative to each other in flexion/extension, rotation, and lateral bending. In turn, this tends to restrict movement of the upper and lower shank members 214 and 220. Because the shank members are coupled to the bone anchors that are coupled to the vertebrae themselves, the vertebrae are also restricted to movement about the center of rotation “A”. This spherical movement about a center of rotation thus tends to approach the natural motion of adjacent vertebrae as they move generally about the center of a healthy, natural disc when cushioned by the disc.

FIGS. 2A and 2B diagrammatically illustrate the generally spherical movement of the pivoted ends 222 and 226 of strips 204 and 206 of the dynamic stabilization device 200 about center of rotation “A” during flexion/extension. FIG. 2B shows the position of the strips 204 and 206 in the generally middle or “neutral” position. This position is in contrast with FIG. 2A, which shows the position of the strips 204 and 204 after flexion/extension, as would occur when a person bends forward.

FIGS. 2C and 2D diagrammatically illustrate one position of the generally spherical movement of the pivoted ends 222 and 226 of strips 204 and 206 of the dynamic stabilization device 200 about the center of rotation “A” during lateral bending. FIG. 2C shows the position of the strips 204 and 206 in the generally middle or “neutral” position and FIG. 2D illustrates the position of the strips 204 and 206 after bending to the right, as would occur when a person bends to the right.

FIGS. 2E and 2F diagrammatically illustrate the generally spherical movement of the pivoted ends 222 and 226 of strips 204 and 206 of the dynamic stabilization device 200 about center of rotation “A” during rotation. FIG. 2E shows the position of the strips 204 and 206 in the generally middle, “neutral” position and FIG. 2F shows the position of the strips 204 and 206 after clockwise rotation, as would occur when a person turns clockwise (i.e., to the right).

FIG. 2G depicts an alternative embodiment of a dynamic stabilization system 240 for both permitting movement between neighboring vertebrae in flexion/extension, lateral bending, and rotation directions, and restraining the degree of movement generally along a curved surface, such as an imaginary spherical shell about a spherical center of rotation “A”. The dynamic stabilization system 240 comprises a first bone anchor 242a, a second bone anchor 242b, and a dynamic stabilization device 243. As illustrated in FIG. 2G, the bone anchors 242a and 242b may be pedicle screws. It is understood that this is but one embodiment of the manner in which a dynamic stabilization system can be employed to partially off-load (or un-weight) the disc between vertebrae (to reduce compression forces) so that as the spine moves through its range of motion pressure on the disc is reduced throughout the entire range of motion. In this embodiment, the bone anchors 242a and 242b may be positioned in the pedicles of the spine as discussed and shown in the above-identified co-pending U.S. patent application Ser. No. 10/690,211, filed on Oct. 23, 2003, entitled “SYSTEM AND METHOD FOR STABILIZING INTERNAL STRUCTURES,” which is incorporated herein by reference.

In certain embodiments, the bone anchors 242a and 242b may include slotted heads 244a and 244b, respectively. In some embodiments, the connection between the bone anchors 242a-242b and the slotted heads 244a-244b may comprise a polyaxial connection. The bone anchors 242a and 242b may be attached to the respective vertebrae (not shown) by screwing the threaded portions 252a and 252b of bone anchors 242a and 242b into the bone of the respective vertebra. Slotted heads 244a and 244b may be respectively coupled at their respective open ends 246a and 246b to an upper attachment member 248 and a lower attachment member 250. The upper and lower attachment members 248 and 250 may have shank portions 249 and 251, respectively. The shank portions 249 and 251 may be placed into the respective open slotted ends 246a and 246b. In certain embodiments, locking elements, such as star-headed locking caps 254a and 254b having helical threads may then be screwed into threaded portions (not shown) of open slotted ends 246a and 246b to lock the shank members 249 and 251 into the open ends 246a and 246b, respectively.

The dynamic stabilization device 243 is conceptually similar to the dynamic stabilization device 200 described in reference to FIGS. 2A-2F. The dynamic stabilization device 243 may also include an elbow 256 having an upper member 258 and a lower member 260 that may be pivotably interconnected at a pivot connection 262. In this exemplary embodiment, an interconnecting end 264 of lower member 260 can be configured as a slotted yoke, where a slot in the middle of the yoke receives an interconnecting end 266 of the upper member 258. The end 266 of upper member 258 may be in the configuration of a flat finger or blade. The interconnecting end 264 of lower member 260 may then be pivotably connected to the interconnecting end 266 of the upper 258 by means of the pivot connection 262 having a pin 263.

In certain embodiments, the upper member 258 may include a rounded upper stop surface 268 that can abut against an upper edge of the lower member 260 when the upper and lower members 258 and 260 of elbow 256 are sufficiently bent. This tends to limit the maximum degree of bending of elbow 256, preventing excessive compression of the disc or disc replacement under conditions of high load. However, in other embodiments, the stop surface 268 can be omitted, if desired.

An outboard end 276 of the upper member 258 may be pivotably connected to the upper attachment member 248. In certain embodiments, the upper attachment member 248 includes a slotted yoke portion 272 and the shank portion 249. The outboard end 276 of the upper member 258 may can be configured as a flat finger which is received by the slotted yoke portion 272. The outboard end 276 can rotate within the slotted yoke portion 272 about a pin 277. Thus, the upper member 258 may be pivotedly connected to the upper attachment member 248. Similarly, an outboard end 286 of the lower member 260 may be pivotably connected to the lower attachment member 250, which includes a slotted yoke portion 282 and the shank portion 251. The outboard end 286 of the lower member 260 may can be configured as a flat finger which is received by the slotted yoke portion 282. The outboard end 286 can rotate within the slotted yoke portion 282 about a pin 287. Thus, the lower member 260 may be pivotedly connected to the lower attachment member 250.

The pins 263, 277, and 287 each have a longitudinal axis that intersects with the others at the center of rotation point “A.” Furthermore, in this embodiment, the elbow 256, the yoke portion 272, and the yoke portion 282 are configured in such a manner that the pin 277 follows a spherical path with respect to the pin 287. The rotational center of the spherical path is the center of rotation “A.” Thus, the dynamic stabilization device 243 has a range of motion similar to the dynamic stabilization device 200 described above with respect to FIGS. 2A-2F.

In certain embodiments, a flexible element, such as a helical spring 288, may be coupled to the dynamic stabilization device 243 in a somewhat compressed condition, whereby it provides a force for providing some degree of distracting and/or unloading of inter-vertebral discs and also allows limited axial and bending movement between the neighboring vertebrae. While various embodiments are described herein as employing a spring for achieving the permissible degree of movement in the dynamic stabilization device, other devices will be readily recognized for substituting for this function, such as an elastomeric sleeve, or a hydraulic, pneumatic or other distracting system.

In the illustrated embodiment, one end of the spring 288 may be inserted into a generally vertical bore (not shown) within the yoke portion 272 of the upper connecting member 248. Similarly, the other end of the spring 288 may be inserted into a generally vertical bore within the yoke portion 282 of the lower connecting member 250.

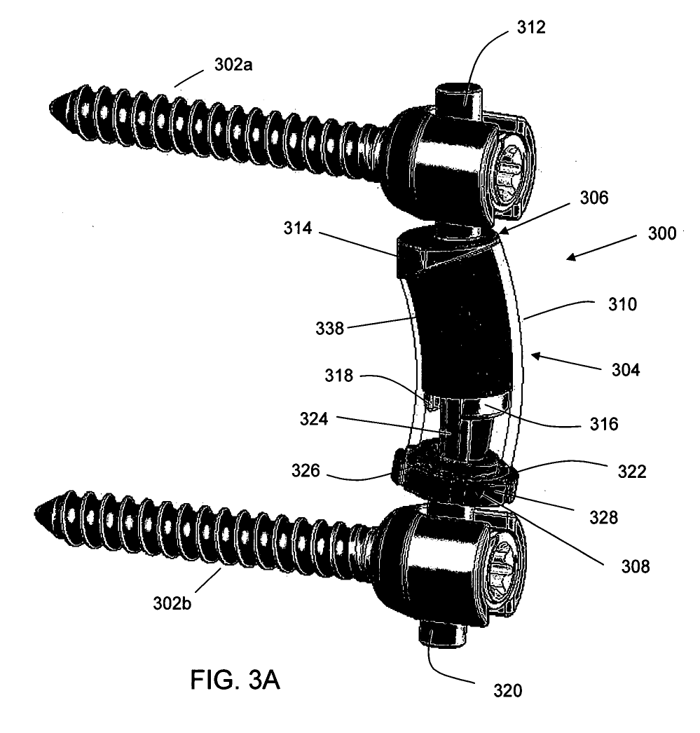

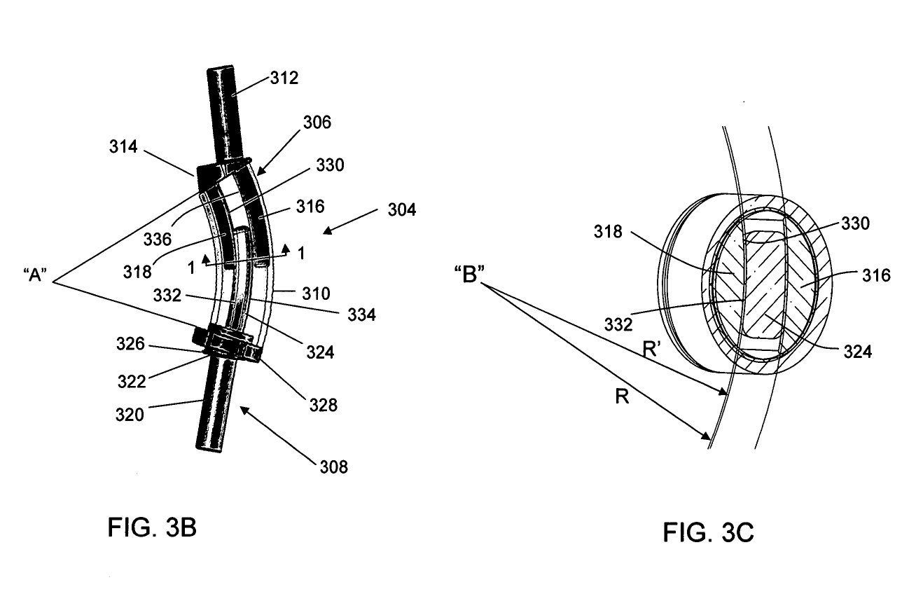

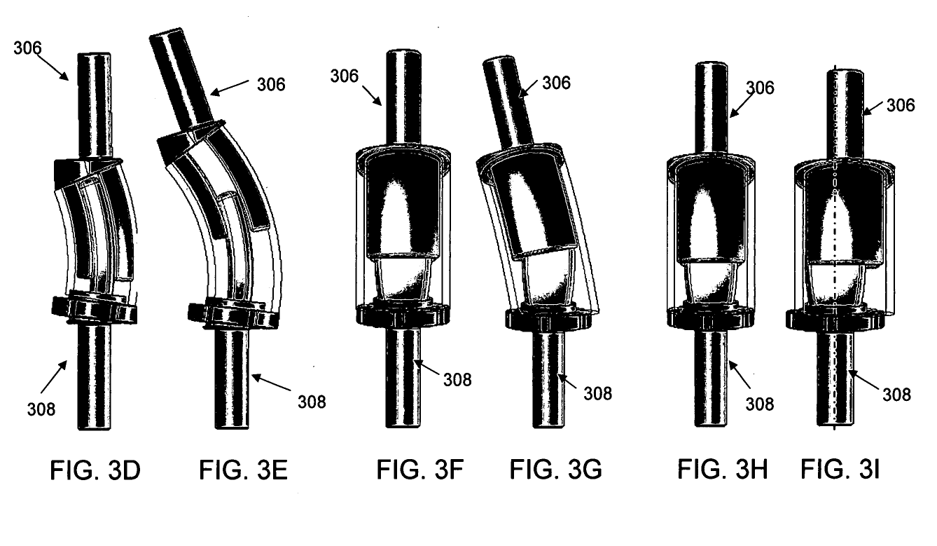

FIG. 3A depicts an alternative embodiment of a dynamic stabilization system 300 for both applying an anterior-posterior distracting force to unload inter-vertebral discs and allowing movement between the neighboring vertebrae. The dynamic stabilization system 300 comprises a first anchor 302a, a second anchor 302b, and a support member or dynamic stabilization device 304. In this exemplary embodiment, the first and second anchors 302a and 302b may be similar to the anchors 242a and 242b described in reference to FIG. 2G. Furthermore, they may be attached to the dynamic stabilization device 304 in a conventional manner or in a manner similar to that described above in reference to FIG. 2G.

In certain embodiments, the dynamic stabilization system 300 creates an anterior distracting force for providing substantially even unloading of inter-vertebral discs, and allows limited movement about an imaginary three dimensional surface (such as a sphere).

FIG. 3B is a section view of the dynamic stabilization device 304 illustrated in FIG. 3A. Turning now to both FIGS. 3A and 3B, there is illustrated the dynamic stabilization device 304 which includes an upper female member 306, a lower male member 308, and a flexible sleeve 310 (which is shown semi-transparent for clarity in FIG. 3A). The flexible sleeve 310 may be an elastomeric sleeve (as illustrated) or a helical spring having a circular or elliptical shape. The upper female member 306 further comprises an upper shank or attachment member 312, an upper collar 314, an outer plate member 316, and an inner plate member 318. The lower male member 308 comprises a lower shank or attachment member 320, a transition portion 322, and a plate member 324.

In the illustrated embodiment, the transition portion 322 may be a threaded portion comprising helical exterior threads 326 that are adapted to mate with a force adjustment ring or sleeve retainer 328. The sleeve retainer 328 may include internal threads that can be cooperatively threaded onto external threads 326 of the lower male member 308. In use, the sleeve retainer 328 restrains the flexible sleeve 310 and provides an adjustable force on the sleeve so that the sleeve may resist compression of the brace 304. The sleeve retainer 328 can be vertically adjusted by rotation about the external threads 326 to vary the compression resistance of the sleeve 310.

With specific reference to FIG. 3B, as previously discussed, the upper female member 306 comprises an outer plate member 316 and an inner plate member 318. In certain embodiments, the lower plate member 324 may be a plate member sized to slideably move between the outer plate member 316 and the inner plate member 318 in both a vertical and a horizontal direction.

In some embodiments, the inner plate member 318 has a curved surface 330 that has a radius centered at point “A.” The lower plate member 324 also has a curved surface 332 that also has a radius centered on a horizontal or X-axis at point “A” such that the curved surface 332 of lower plate member 324 may slidingly engage the curved surface 330. In some embodiments, the lower plate member 324 may also have a curved surface 334 that slidingly engages a curved surface 336 of the outer plate member 316. With respect to the vertical movement or components of the vertical movement, the curved surfaces 330, 332, 334, and 336 of the plate members 316, 324, and 318 have radii which are centered about point “A.” Thus, when viewed from the perspective of FIG. 3B, the lower plate member 324 may move or rotate about the center point “A” with respect to the two plate members 316 and 318.

FIG. 3C is an isometric section view cut through the dynamic stabilization device 304 at a line 1-1 on FIG. 3B. As illustrated, the lower plate member 324 is sandwiched between the outer plate member 316 and the inner plate member 318. As illustrated in this embodiment, the curved surface 330 of the inner plate member 318 is also curved about a vertical or Y-axis having a radius of curvature R that is centered at point “B.” Similarly, the curved surface 332 of the lower plate member 324 is also curved about the y-axis and has a radius of curvature R′ centered at point “B” such that the curved surface 332 of lower plate member 324 may slidingly engage the curved surface 330. In some embodiments, the curved surface 334 of lower plate member 324 may slidingly engage the curved surface 336 of the outer plate member 316. Consequently, the lower plate member 324 may be restricted to a curved horizontal movement with respect to the inner plate member 318 and outer plate member 316.

If point “A” of FIG. 3B and point “B” of FIG. 3C are located substantially at the same point, then the respective surfaces may be spherical. In other words, if the radii of curvature for the surface of the plate members have a common center about all axes or directions, then the surfaces would be spherical surfaces. In other words, the surfaces of the plate members may be thought of as spherical surfaces which slide over each other. Thus, the dynamic stabilization device 304 has a motion similar to the dynamic stabilization device 200 described above with respect to FIGS. 2A-2F. The range of movement of the dynamic stabilization device 304 may be more limited than the range of movement of the dynamic stabilization device 200 due to the size of the respective plates.

Referring again to FIG. 3A, in some embodiments, there is an inner fabric sleeve 338 which laterally restrains the lower male member 308 relative to the upper female member 306. This inner fabric sleeve 338 may be made of a surgical fabric or another braided material.

FIG. 3D illustrates in a sagittal (side) view the relative positions of the upper female member 306 and the lower male member 308 in an extension position. In contrast, FIG. 3E illustrates the relative positions of the upper female member 306 and the lower male member 308 during flexion.

FIG. 3F illustrates in a posterior view the relative positions of the upper female member 306 and the lower male member 308 in a normal, undisplaced position at rest. In contrast, FIG. 3G illustrates the relative positions of the upper female member 306 and the lower male member 308 during lateral bending of the spine.

FIG. 3H illustrates in a posterior view the relative positions of the upper female member 306 and the lower male member 308 when in a normal, undisplaced position at rest. In contrast, FIG. 3J illustrates the relative positions of the upper female member 306 and the lower male member 308 during axial rotation of the spine.

Thus, this embodiment of the dynamic stabilization device 304 provides movement in three degrees of freedom, particularly with respect to flexion/extension, lateral bending, and rotation, so that as the spine moves through its normal range of motion, pressure on the disc between adjacent vertebrae is reduced throughout the range of motion.

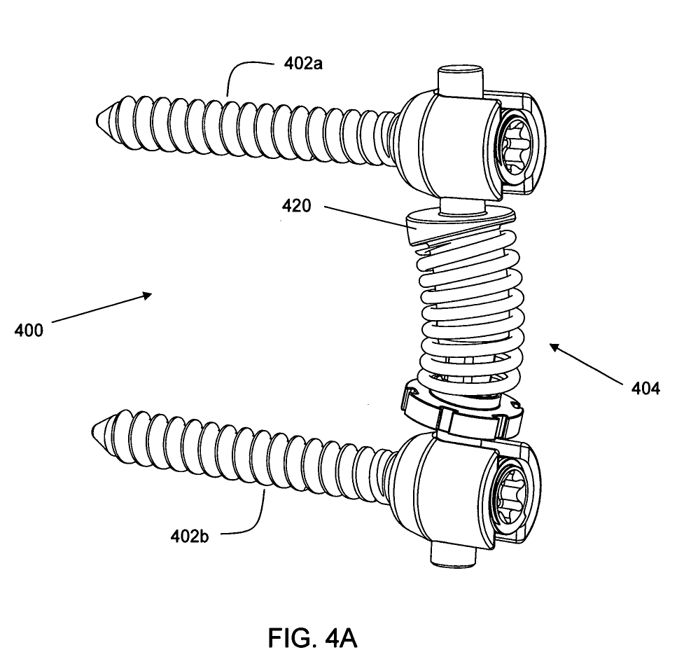

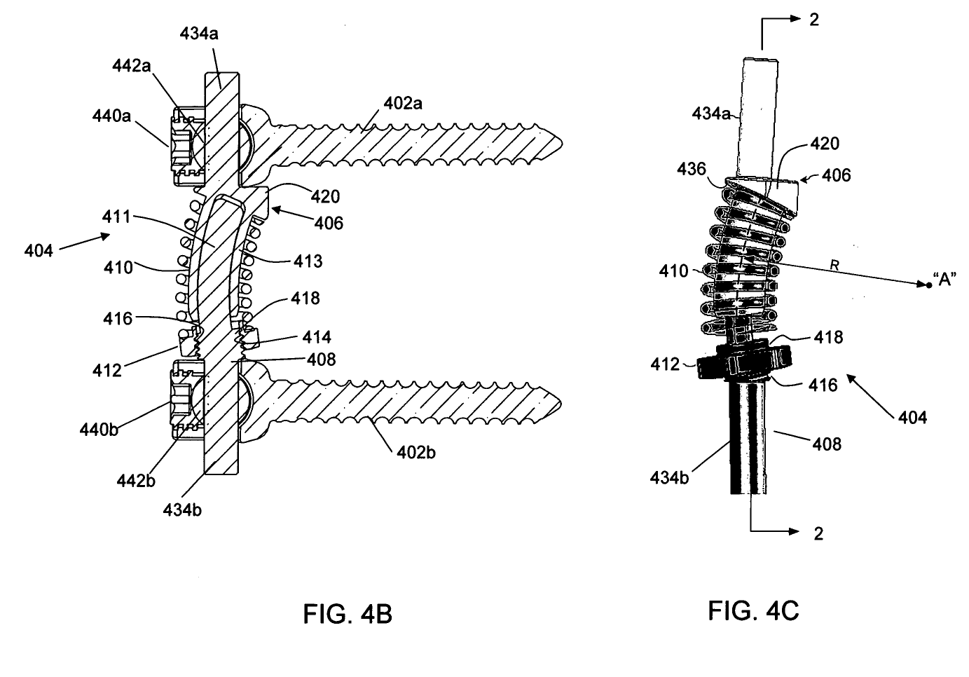

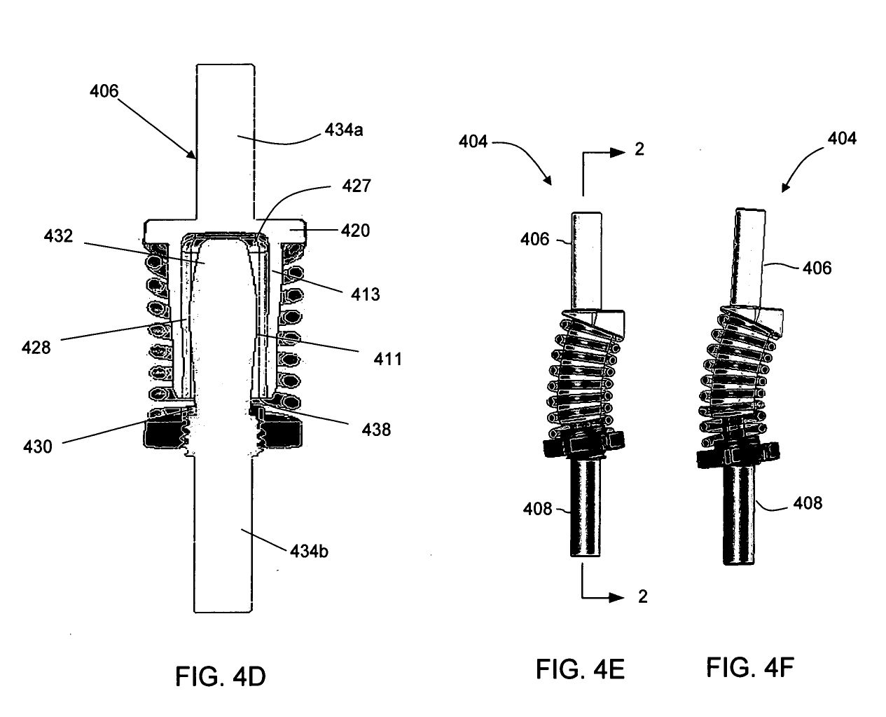

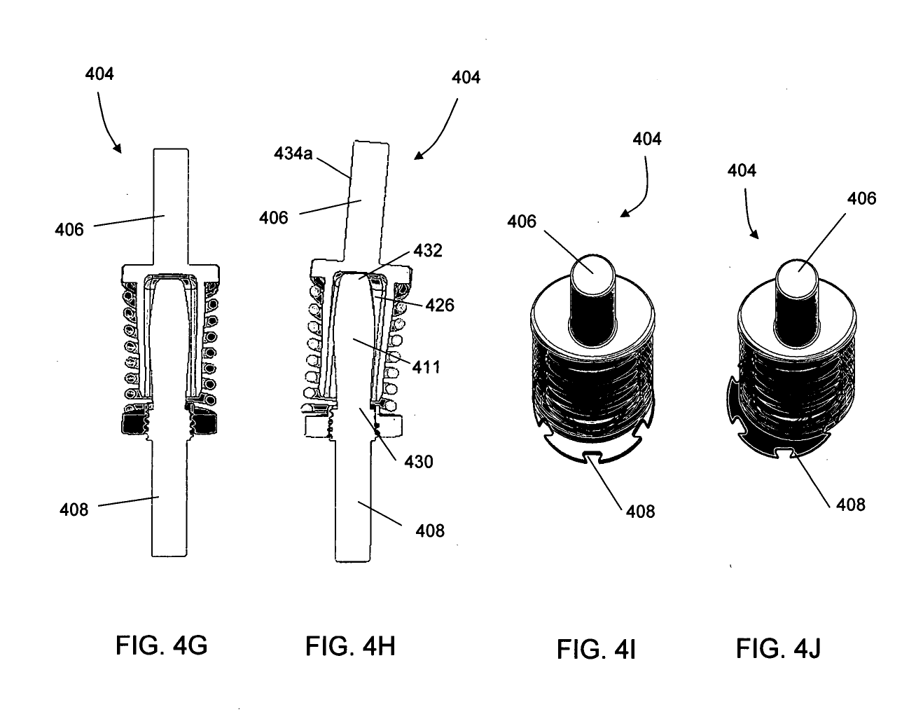

FIG. 4A is an isometric view of another embodiment of a dynamic stabilization system 400 for both applying an anterior-posterior distracting force to unload inter-vertebral discs and allowing movement between the neighboring vertebrae. In certain embodiments, the dynamic stabilization system 400 creates an anterior distracting force for providing substantially even unloading of inter-vertebral discs, and allows limited movement about an imaginary two dimensional or three dimensional curved surface (such as a sphere between the neighboring vertebrae).

FIG. 4B is a section view of the dynamic stabilization system 400. Referring to both FIG. 4A and FIG. 4B, in this embodiment, the dynamic stabilization system 400 comprises a first anchor 402a, a second anchor 402b, and a dynamic stabilization device 404. In this exemplary embodiment, the first and second anchors 402a and 402b are similar to the anchors 242a and 242b described in reference to FIG. 2G. Furthermore, they may be attached to the dynamic stabilization device 404 in a conventional manner or in a manner similar to that which is described above in reference to FIG. 2G. For instance, locking caps 440a-440b may have a curved surface adapted to engage ball shaped members 442a-442b. When the caps 440a-440b are screwed down, a force is exerted on the ball shaped member 442a-442b. The ball shaped member may have a notched portion, which would then fail under pressure causing the ball shaped member to engage the surface of shank portions 434a-434b of the dynamic stabilization device 404. As force is exerted on the ball shaped members 442a-442b by the locking members, the ball shaped members 442a-442a may also engage the interior surface of the anchor heads, thereby fixing the shank members and the ball members in place.

With additional reference to FIG. 4C (which is a side view of the dynamic stabilization device 404), it can be seen that the dynamic stabilization device 404 may comprise an upper guide member 406, a lower post member 408, a spring member 410, an upper stop 420, and a spring retainer 412. In some embodiments, the lower post member 408 may include a post portion 411 that may be curved along its length at a radius of curvature R which has a center about point “A.” In some embodiments, the post portion 411 may also be curved in a generally transverse direction from its longitudinal axis. Such a curve may follow a second radius of curvature, which may or may not be the same radius of curvature as the radius of curvature R. Such a curve would allow the post portion 411 to rotate about the vertical axis in a manner similar to that described in reference to FIGS. 3A-3F. In yet other embodiments, the lower post member may be generally round or rectangular in cross-section about its axis.

The post portion 411 may fit inside a guide portion 413 of the upper guide member 406. In the illustrative embodiment, both portions are curved. It is this curve that allows the bone anchor 402a to move in an arc when the pedicle to which the bone anchor 402b is attached rotates in flexion. This allows the dynamic stabilization device 400 to rotate about a center of rotation with a curved motion. Note that the X-axis center of rotation of dynamic stabilization device 400 is controlled by the bend of the post portion 411 relative to the guide portion 413.

In this embodiment, the radius of curvature R may inscribe a path that approximately corresponds to the path followed by the middle of the post portion 411 when the person bends, thus angularly displacing the upper adjacent vertebrae with respect to the lower vertebra. The path followed by the center line of the post portion 411 constrains and guides relative rotation of the posterior portions of the upper and lower vertebrae about one or more horizontal axes of rotation in the vicinity of the center of radius of curvature R. In some embodiments, one or more axes of rotation are located near or coincide with the axes of rotation of the upper and lower vertebrae in a healthy and undamaged spine.

The spring member 410 introduces an increasing resistance to further retraction or extension as a limit of practical or permissible movement is approached. The spring member 410 may be positioned around the outside of the upper guide member 406 between the upper stop 420 and the spring retainer 412. The spring retainer 412 may include internal threads 414 that can be cooperatively threaded onto external threads 416 of connecting portion 418 of the lower post member 408 to retain the spring member 410 and to provide a force urging extension of the dynamic stabilization device 404. In certain embodiments, the spring retainer 412 can be vertically adjusted by rotation about the external threads 416 to vary the compression of the spring 410 and the resulting force of the spring 410 urging upper guide member 406 and lower post member 408 apart. In certain embodiments, the spring 410 may be held in compression and may be adjusted by the rotatable spring retainer 412 moving under control of a set of interior threads.

FIG. 4D depicts the dynamic stabilization device 404 in a cross-sectional, coronal view, taken along the line 2-2 of FIG. 4C In the illustrative embodiment, the post portion 411 may be somewhat wider in a generally medial portion 428 than at either a root 430 or end portion 432. In certain embodiments, the guide portion 413 of the upper guide member 406 may have an elongated hole 427 with an open end 438. The elongated hole 427 may generally be curved along its length to approximately match the radius of curvature of the lower post member 411, and having internal dimensions just slightly larger than the cross-sectional dimensions of the generally medial portion 428 of the post portion 411. Thus, significant clearance may exist between the post portion 411 and the internal walls of the guide portion 413, above and below the generally medial portion 428. The post portion 411 can, therefore, be angularly displaced with respect to guide portion 413 of the upper guide member 406 to the extent of the clearance, as well as being free to rotate within the guide portion 413 of the upper guide member 406. Furthermore, the post portion 411 is also free to be longitudinally displaced with respect to guide portion 413 to the degree permitted by spring 410.

Thus, the brace 404 provides movement in three degrees of freedom, particularly with respect to flexion/extension, lateral bending, and rotation, so that as the spine moves through its normal range of motion, pressure on the disc between adjacent vertebrae is reduced throughout the entire range of motion.

Referring again to FIG. 4C, the spring 410 may be confined at its upper end by the stop 420, located between an upper shank portion 434a and the guide portion 413. In some embodiments, the stop 420 may have a slanted shoulder 436, against which the spring 410 abuts. The spring 410, the upper guide member 406, and the post portion 411 of the lower post member 408 may be arched somewhat away from the vertebrae, thus providing clearance from the vertebrae. This tends to provide a stable position of the completed structure (including both support members mounted to the adjacent vertebrae) when the vertebrae are in the approximately middle, undisplaced position. If desired, the open end 438 of the upper guide member 406 can be somewhat smaller than the maximum diameter of the medial portion 428 of the post portion 411. This will prevent the post portion 411 from pulling out of the upper guide member 406 completely in the event of hyperextension.

FIG. 4E illustrates a sagittal (side) view of the relative positions of upper guide member 406 and lower post member 408 in a normal, retracted position while at rest, whereas FIG. 4F illustrates the relative positions of upper guide member 406 and lower post member 408 in an extended position during flexion/extension.

FIG. 4G illustrates in a coronal (front) view of the relative positions of upper guide member 406 and lower post member 408 in a normal, undisplaced position while at rest, whereas FIG. 4H illustrates the relative positions of upper guide member 406 and lower post member 408 in an angularly skewed position during lateral bending. It should be noted that the angular skewing of the brace 404 is constrained within a desired range of motion by the degree of clearance between the interior walls of upper guide member 406 and the root 430 and end portion 432 of the post portion 411. Twisting of the post portion 411 within the guide portion 413 need not be limited, however, because at least a pair of dynamic stabilization devices 404 are typically used. The use of at least two of the dynamic stabilization devices 404 between adjacent vertebrae, with each upper and lower shank portion 434a and 434b of each dynamic stabilization device 404 fixedly attached to adjacent vertebrae, limits twisting of the lower post member 408 within the upper guide member 406 to a desired degree.

FIG. 4I illustrates a somewhat oblique, upper view of the upper end of the dynamic stabilization device 404, showing the relative positions of lower post member 408 and upper guide member 406 in a normal, retracted position while at rest, whereas FIG. 4J illustrates the relative positions of lower post member 408 and upper guide member 406 in a sidewise-displaced condition during rotation.

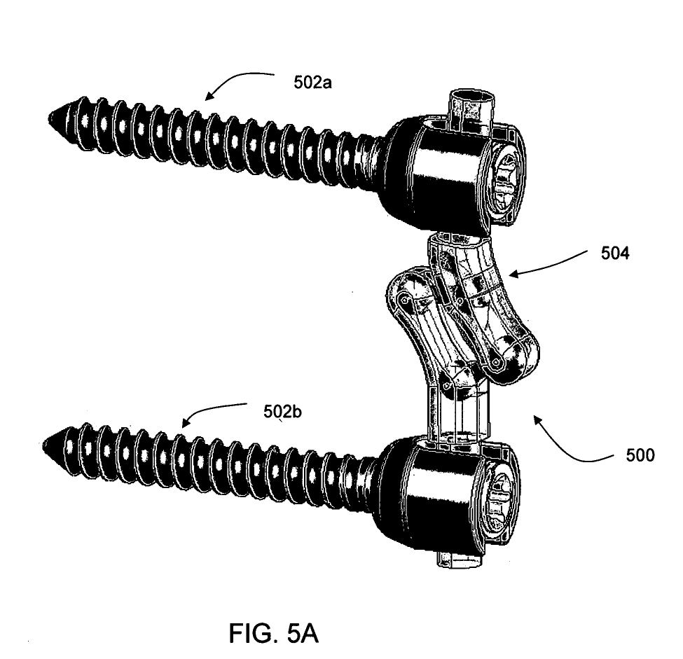

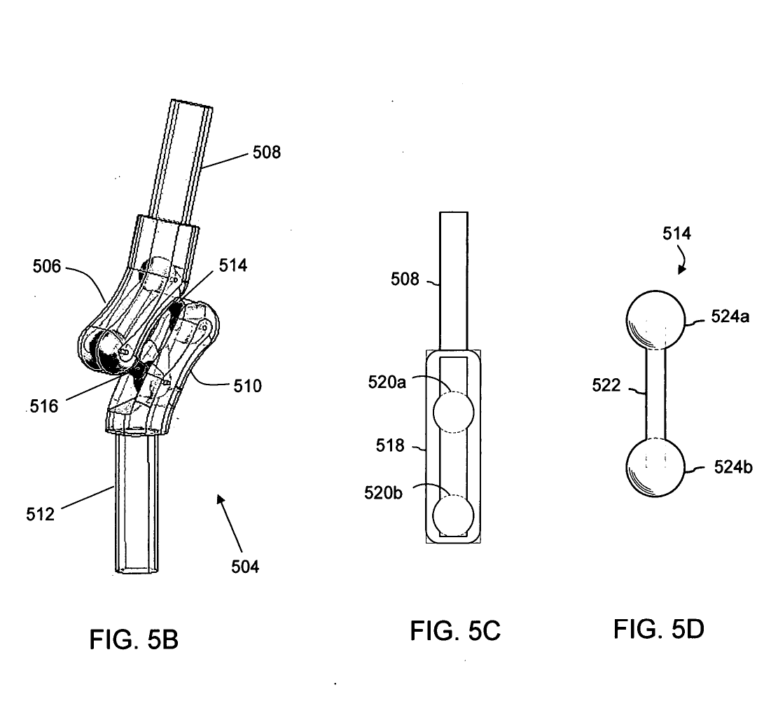

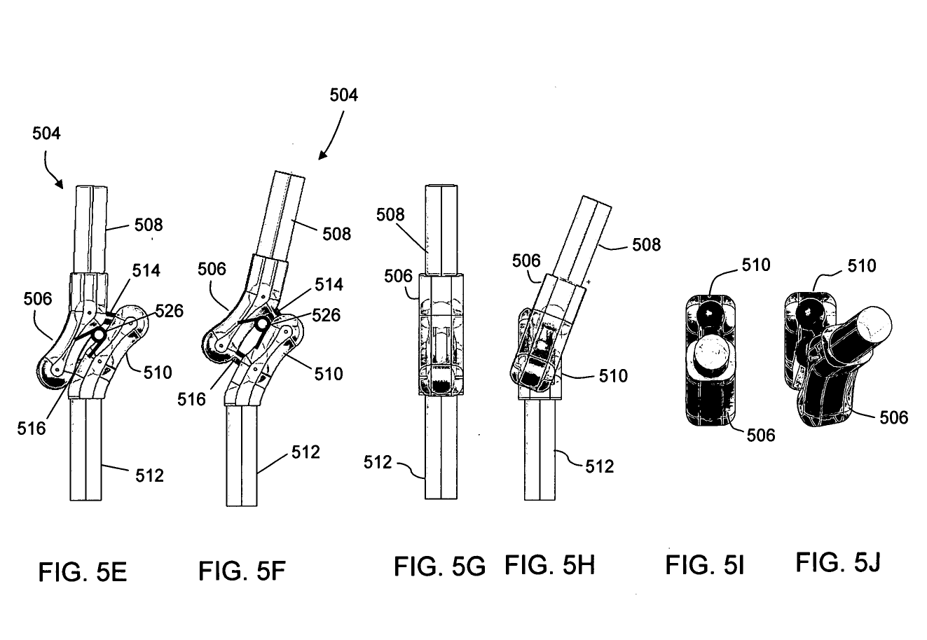

FIG. 5A is an isometric view of another embodiment of a dynamic stabilization system 500 for both applying an anterior-posterior distracting force to unload inter-vertebral discs and allowing movement between the neighboring vertebrae. The dynamic stabilization system 500 comprises a first anchor 502a, a second anchor 502b, and a dynamic stabilization device 504. In this exemplary embodiment, the first and second anchors 502a and 502b may be similar to the anchors 242a and 242b described in reference to FIG. 2G. Furthermore, they may be attached to the dynamic stabilization device 504 in a conventional manner or in a manner similar to that which is described above in reference to FIG. 2G.

In certain embodiments, the dynamic stabilization system 500 creates an anterior distracting force for providing substantially even unloading of inter-vertebral discs, and allows limited movement about an imaginary two dimensional or three dimensional curved surface.

FIG. 5B is a detailed isometric view of the dynamic stabilization device 504. As illustrated, the dynamic stabilization device 504 may comprise an upper connecting member 506 coupled to an upper shank member 508, a lower connecting member 510 coupled to a lower shank member 512, a first coupler member 514 and a second coupler member 516 interlinked with the upper and lower connecting members for movement, and one or more spring members (not shown) providing a force for controlling the movement between the upper connecting member 506 and the lower connecting member 510. Each coupler member 514, 516 may be rotatably connected at either end thereof to one of the connecting members 506, 510 to form a flexible, trapezoidal linkage. The various components of dynamic stabilization device 504 are configured to permit movement of the dynamic stabilization device in three degrees of freedom.

FIG. 5C is a section view cut longitudinally along the axis of the upper connecting member 506. In this embodiment, the upper connecting member 506 comprises a yoke portion 518 and the shank portion 508. The lower connecting member 510 may be similarly constructed. As described previously, each connecting member 506, 510 can be secured to one of the anchors 502a and 502b at the shank portion 508, 512. In certain embodiments, the yoke portion 518 may include semi-spherical cavities 520a and 520b for receiving an end of one of the coupler members 514, 516.

With additional reference to FIG. 5D, an illustration of one embodiment of a coupler member (e.g., the coupler member 514 of FIG. 5C) is provided. Each coupler member 514, 516 comprises a shank portion 522, a first spherical portion 524a, and a second spherical portion 524b. A spherical portion 524a or 524b of coupler member 514, 516 is inserted into and captured by one of the spherical cavities 520a or 520b (FIG. 5C) in the yoke portion 518 of each connecting member 506, 510 to form the four-bar dynamic stabilization device 504 having a variable trapezoidal geometry that tilts the upper shank portion 508 forward relative to the lower shank portion 512 as the dynamic stabilization device extends.

Relative extension, retraction, rotation and skewing of the connecting members 506, 510 of the dynamic stabilization device 504 are constrained within a desired range of motion by the coupler members 514, 516, which in turn have a limited range of pivot caused by the apertures of their respective sockets formed by the spherical cavities 520a, 520b. The rims of the spherical cavities 520a, 520b abut the shanks of the coupler members 514, 516 to limit the range of motion. Alternatively or additionally, one or more stops can be formed on the surfaces of the connecting members 506, 510 to limit the range of movement of the interconnecting coupler members 514, 516.

Dynamic stabilization device 504 allows for movement in three degrees of freedom, particularly with respect to flexion/extension, lateral bending, and rotation, so that as the spine moves through its normal range of motion, pressure on the disc between adjacent vertebrae is reduced throughout the range of motion. As shown in sagittal (side) view in FIGS. 5E and 5F, coupler members 514, 516 may rotate to permit connecting member 506 to extend or move upwardly with respect to connecting member 510.

FIG. 5E illustrates the relative positions of connecting members 506, 510 in a normal, retracted position while at rest, whereas FIG. 5F illustrates the relative positions of connecting members 506, 510 in an extended position while in flexion or extension. In some embodiments, a distracting mechanism, such as one or more resilient spring members, for instance a torsional spring 526 shown in FIGS. 5E and 5F, urge the connecting members 506, 510 apart. The spring 526 may also increase resistance to further retraction as the connecting members retract. Surfaces of connecting members 506, 510 can abut to limit retraction of the brace 504, and surfaces of coupler members 514, 516 can abut surfaces at the edges of spherical cavities 520a, 520b to limit extension and/or retraction. Rotation or pivoting of the spherical portions 524a, 524b of coupler members 514, 516 within the spherical cavities 520a, 520b of connecting members 506, 510 permit movement of connecting members 506, 510 away from or toward each other as required in flexion/extension as a person bends forwards or backwards at the waist.

Referring to FIGS. 5G and 5H, the structural configuration of the connecting members 506, 510 and the coupler members 514, 516 may enable movement of the dynamic stabilization device 504 in lateral bending. Coupler members 514, 516 may rotate or pivot laterally with respect to connecting members 506, 510, thereby allowing limited lateral bending movement. FIG. 5G illustrates the relative positions of connecting members 506, 510 in a normal position while at rest, whereas FIG. 5H illustrates the relative positions of connecting members 506, 510 in a laterally bent position. Surfaces of connecting members 506, 510 can abut as the limit of lateral bending is reached, and surfaces of coupler members 514, 516 can abut surfaces at the edges of spherical cavities 520a, 520b to prevent further lateral bending. Rotation or pivoting of the spherical portions 524a, 524b of coupler members 514, 516 within the spherical cavities 520a, 520b of connecting members 506, 510 permit lateral pivotal movement or rotation of connecting member 506 with respect to connecting member 510, as required in lateral bending as a person bends sideways.

As shown in FIGS. 5I and 5J, the structural configuration of the connecting members 506, 510 and the coupler members 514, 516 may also allow movement of the dynamic brace 504 in rotation. Coupler members 514, 516 pivot with respect to connecting members 506, 510, thereby allowing connecting members 506, 510 to rotate with respect to each other. FIG. 5I illustrates the relative positions of connecting members 506, 510, in a normal position while at rest, whereas FIG. 5J illustrates the relative positions of connecting members 506, 510 in rotation. Surfaces of connecting members 506, 510 can abut as the limit of rotation is reached, and surfaces of coupler members 514, 516 can abut surfaces at the edges of spherical cavities 520a, 520b to prevent further rotation. Rotation or pivoting of the spherical portions 524a, 524b of coupler members 514, 516 within the spherical cavities 512, 520b of connecting members 506, 510 permits rotation of connecting member 506 with respect to connecting member 510 as required when person rotates their torso to the left or to the right.

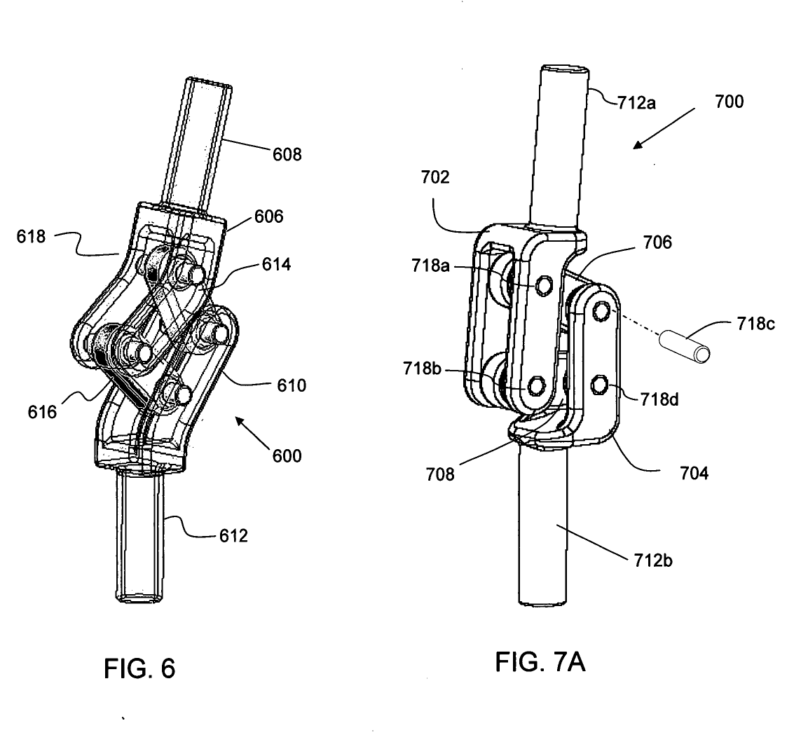

FIG. 6 is an isometric drawing illustrating another embodiment of a four-bar dynamic stabilization device 600 that is conceptually similar to the dynamic stabilization device 504 described with reference to FIG. 5A. In certain embodiments, the dynamic stabilization device 600 creates an anterior distracting force for providing substantially even unloading of inter-vertebral discs, and allows limited movement about an imaginary two dimensional or three dimensional curved surface.

The dynamic stabilization device 600 may comprise an upper connecting member 606 coupled to an upper shank member 608, a lower connecting member 610 coupled to a lower shank member 612, a first coupler member 614 and a second coupler member 616 interlinked with the upper and lower connecting members for movement, and one or more spring members (not shown) providing a force for controlling the movement between the upper connecting member 606 and the lower connecting member 610. Each coupler member 614, 616 is rotatably connected at either end thereof to one of the connecting members 606, 610 to form a trapezoidal linkage.

In this embodiment, the upper connecting member 606 comprises a yoke portion 618. The lower connecting member 610 is similarly constructed. Each coupler member 614, 616 may have end bearing connections that allow rotation about three degrees of freedom in a manner similar to the dynamic stabilization device 504 discussed in reference to FIGS. 5A-5J.

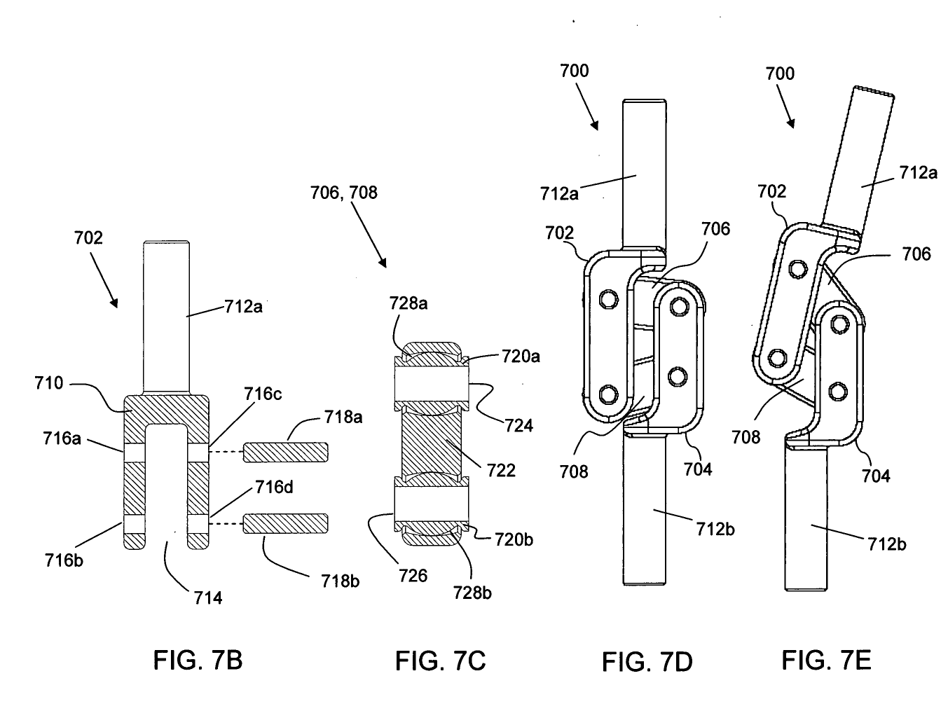

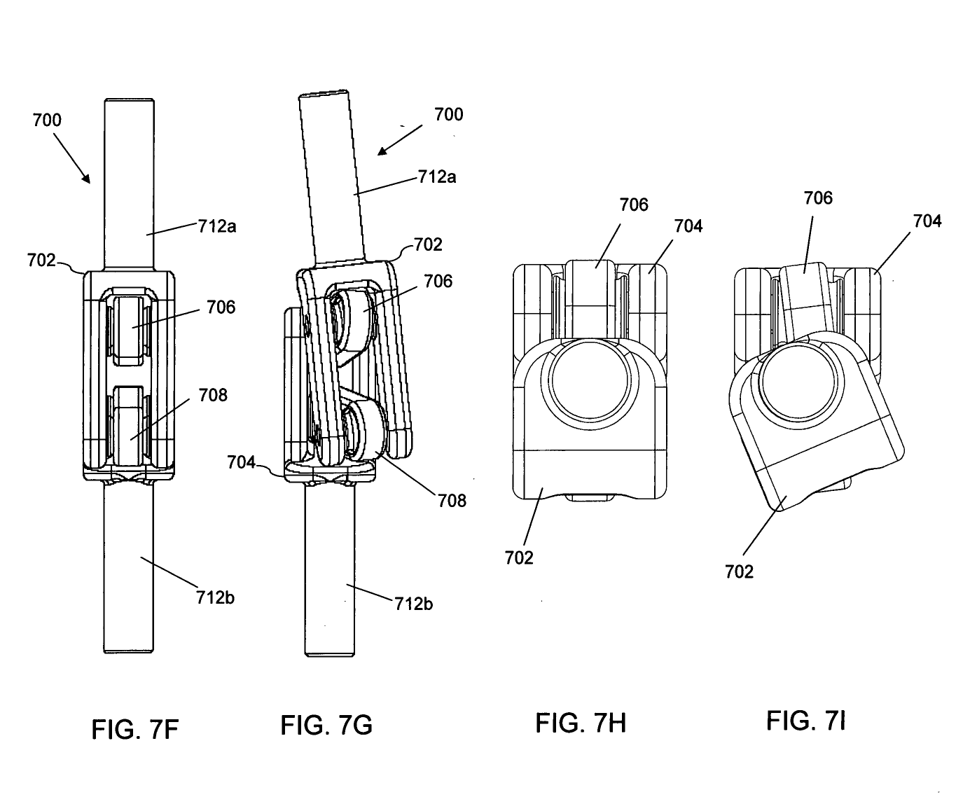

Referring to FIG. 7A, another embodiment of a four-bar dynamic stabilization device 700 is illustrated. The dynamic stabilization device 700 may be conceptually similar to the dynamic stabilization device 600 of FIG. 6. However, the dynamic stabilization device 700 may be configured to achieve movement while maintaining a relatively compact form factor throughout its range of motion. The dynamic stabilization device 700 comprises an upper connecting member 702, a lower connecting member 704, a first coupler 706, and a second coupler 708. As with the dynamic stabilization devices 600 and 504 (discussed in reference to FIGS. 6 and 5A, respectively), the upper and lower connecting members 702, 704 may be interlinked in a manner that will allow relative rotational movement. One or more spring members (not shown) may provide a force for controlling the movement between connecting member 702 and connecting member 704. Connecting pins 718a-d pivotally and rotatably connect the ends of the couplers 706, 708 to one of the connecting members 702, 704 to form the dynamic stabilization device 700, and provide a variable trapezoidal geometry that tilts the upper connecting member 702 relative to the lower connecting member 704 as the dynamic stabilization device extends.

Relative extension, retraction, rotation and skewing of the connecting members 702, 704 of the dynamic stabilization device 700 are constrained within a desired range of motion by the couplers 706 and 708, which in turn have a limited three dimensional range of pivot caused by the use of rod end bearings (FIG. 7C). Dynamic stabilization device 700 provides movement in three degrees of freedom, particularly with respect to flexion/extension, lateral bending, and rotation, so that as the spine moves through its normal range of motion, pressure on the disc between adjacent vertebrae is reduced throughout the range of motion.

FIG. 7B illustrates a section view of one of the connecting members, for instance upper connecting member 702. Upper connecting member 702 comprises a yoke portion 710 and a shank portion 712a. In some embodiments, the upper connecting member 702 can be secured to a bone anchor at the shank portion 712a. Yoke portion 710 includes a slot 714 for receiving an end of each of the couplers 706, 708, and may further include four circular apertures 716a-716d for receiving connecting pins 718a and 718b used to rotatably secure the couplers 706, 708 to the yoke portion 710 of the connecting members 702, 704.

With additional reference to FIG. 7C, a section view of an exemplary coupler (e.g., the coupler 706 of FIG. 7A) is provided. Each connecting pin 718a and 718c may be coupled to a spherical bearing 720a and 720b centrally positioned within the coupler 706. Thus, the bearings 720a-720b may be receive the shafts of the associated connecting pins 718a and 718c. As illustrated, the coupler 706 comprises an elongated body 722 having a first aperture 724 formed transversely through one end thereof and a second aperture 726 formed transversely through the other end thereof. Apertures 724 and 726 each have concave, spherical bearing surfaces 728 at least partially surrounding and having curvature similar to the bearings 720a and 720b. When assembled, the bearings 720a-720b and bearing surfaces 728a-728b of the coupler 706 form rod end bearings that provide lateral pivoting movement and skew movement for the dynamic stabilization device 700 when in lateral bending and/or rotation of the spine. It is understood that the coupler 708 may be constructed in an identical or similar manner.

Each end of couplers 706, 708 may be inserted into the slot 714 of each connecting members 702, 704 to form a rod end bearing with one of the connecting elements 718. Accordingly, the four-bar dynamic stabilization device 700 may be formed having a variable trapezoidal geometry (FIG. 7A) that tilts the upper shank portion 712a forward relative to the lower shank portion 712b as the dynamic stabilization device 700 extends.

The dynamic stabilization device 700 provides movement in three degrees of freedom, particularly with respect to flexion/extension and lateral bending, so that as the spine moves through a curved range of motion, pressure on the disc between adjacent vertebrae is reduced throughout the entire range of motion. As shown in sagittal (side) view in FIGS. 7D and 7E, couplers 706, 708 rotate to permit upper connecting member 702 to extend or move upwardly with respect to lower connecting member 704. FIG. 7D illustrates the relative positions of connecting members 702, 704 in a normal, retracted position while at rest, whereas FIG. 7E illustrates the relative positions of connecting members 702, 704 in an extended position while in flexion or extension. In some embodiments, a distracting mechanism, such as a torsional spring (not shown) may urge the connecting members 702, 704 apart. The spring may also increase resistance to further retraction as the connecting members 702, 704 retract. In some embodiments, surfaces of connecting members 702, 704 can abut to limit retraction of the brace 700, and surfaces of couplers 706, 708 can abut surfaces of connecting members 702, 704 to limit extension and/or retraction. Rotation or pivoting of the couplers 706, 708 around the connecting pins 718a-d permits movement of connecting members 702, 704 away from or toward each other as required in flexion/extension when a person bends forwards or backwards at the waist.

Referring to FIGS. 7F and 7G, the structural configuration of the connecting members 702, 704 and the couplers 706, 708 may also enable movement of the dynamic stabilization device 700 in lateral bending. Couplers 706, 708 may rotate or pivot laterally with respect to connecting members 702, 704, thereby allowing limited lateral bending movement. FIG. 7F illustrates the relative positions of connecting members 702, 704 in a normal position while at rest, whereas FIG. 7G illustrates the relative positions of connecting members 702, 704 in a laterally bent position. Surfaces of connecting members 702, 704 can abut as the limit of lateral bending is reached, and surfaces of couplers 706, 708 can abut with surfaces of connecting members 702, 704 to prevent further lateral bending. Rotation or pivoting of the couplers 706, 708 around the connecting elements 718a-d permits lateral pivotal movement or rotation of connecting member 702 with respect to connecting member 704 as required in lateral bending when a person bends sideways.

As shown in FIGS. 7H and 7I, the structural configuration of the connecting members 702, 704 and the couplers 706, 708 may also allow movement of the dynamic stabilization device 700 in rotation. Couplers 706, 708 may pivot with respect to connecting members 702, 704, thereby allowing connecting members 706, 708 to rotate with respect to each other. FIG. 7H illustrates the relative positions of connecting members 702, 704 in a normal position while at rest, whereas FIG. 7I illustrates the relative positions of connecting members 702, 704 in rotation. Surfaces of connecting members 702, 704 can abut as the limit of rotation is reached, and surfaces of couplers 706, 708 can abut surfaces of connecting members 702, 704 to prevent further rotation. Rotation or pivoting of the couplers 706, 708 around the connecting elements 718a-d permits rotation of connecting member 702 with respect to connecting member 704 as required when a person rotates their torso to the left or to the right.

Use of Multiple Devices in a Single System:

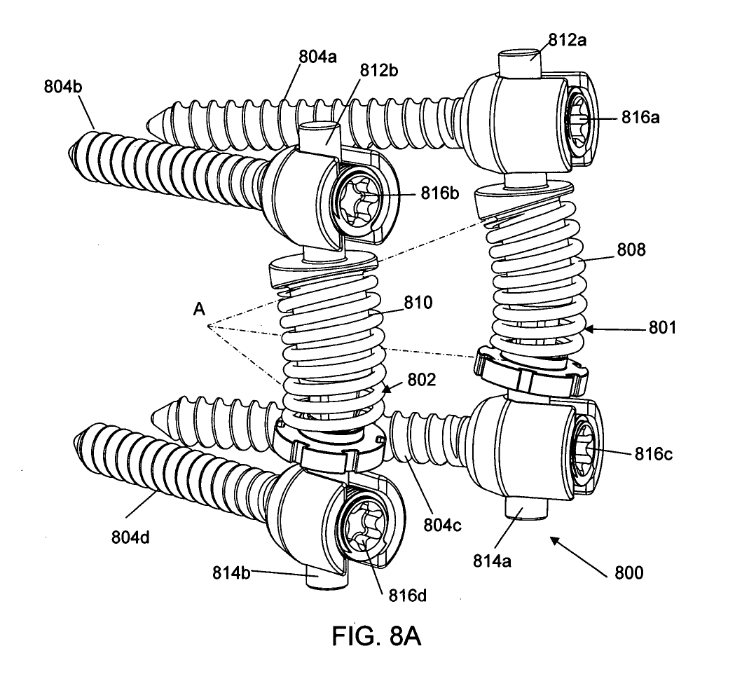

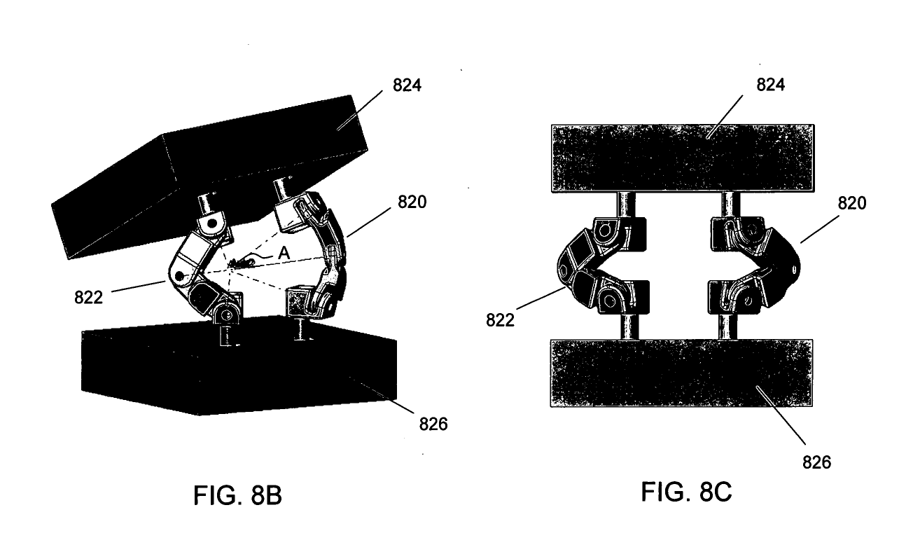

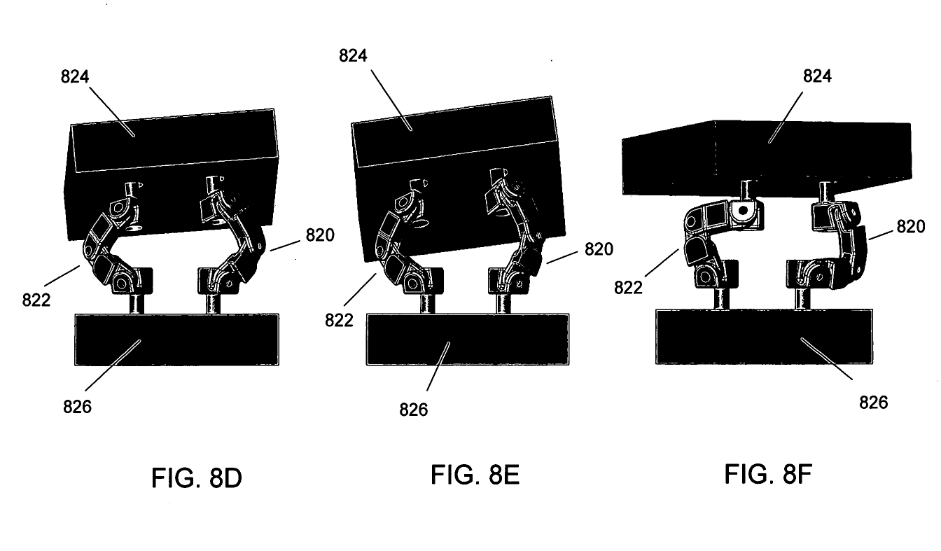

The preceding paragraphs described several embodiments and aspects of single dynamic stabilization systems and devices that enable three dimensional movement. In use, the dynamic stabilization devices may be used in pairs, such as illustrated in FIG. 8A.

FIG. 8A is an isometric view of one embodiment of a system comprising a first dynamic stabilization system 801 and a second dynamic stabilization system 802. The dynamic stabilization systems 801 and 802 may be used together as a single system for both applying an anterior-posterior distracting force to unload inter-vertebral discs and allowing movement between the neighboring vertebrae. Each of the dynamic stabilization systems 801 and 802 may comprise a first or upper anchor 804a and 804b, a second or lower anchor 804c and 804d, and a dynamic stabilization device 808 and 810, respectively. In this exemplary embodiment, the anchors 804a-804d are similar to the anchors 242a and 242b described in reference to FIG. 2G. Furthermore, they may be attached to their respective dynamic stabilization device 808, 810 in a conventional manner or in a manner similar to that described above in reference to FIG. 2G.

Although the dynamic stabilization devices 808, 810 are illustrated in FIG. 8A as slider type devices, these devices are but examples. Any of the dynamic stabilization devices disclosed herein or any combination of dynamic stabilization devices may be used in a similar manner.

The first and second dynamic systems 801 and 802 may be coupled to adjacent upper and lower vertebrae on either side of the corresponding spinous processes in a conventional manner. In the present example, the first anchor 804a couples the first dynamic stabilization device 808 to an upper vertebra (not shown) at its right-hand pedicle. Similarly, the second anchor 804c couples the first dynamic stabilization device 808 to a lower vertebra (not shown) at its right-hand pedicle. A similar procedure may be repeated on the left side of the spinous process where the third anchor 804b couples the second dynamic stabilization device 810 to the upper vertebra by threading into the upper vertebra at its left-hand pedicle. Finally, the fourth anchor 804d threads into the lower vertebra at its left hand pedicle which secures the second dynamic stabilization device 810 to the lower vertebra.

The dynamic stabilization devices 808 and 810 have upper shank portions 812a, 812b and lower shank portions 814a, 814b, respectively. As described above, the shank portions may be secured to the anchors by fasteners, such as set screws 816a-816d. In some embodiments, the upper and lower shank portions 814a, 814b, 812a, and 812b are cylindrical and of uniform diameter. This configuration allows each of the shank portions to slide freely within the respective slotted end portions of their respective pedicle anchors 804a-804d prior to tightening the associated set screws 814a-814d at the desired location along the length of each of the upper and lower shanks.

In certain embodiments, the dynamic stabilization devices are each positioned so that the individual center of rotation for each device may be centered at a common point “A.” This positioning allows both dynamic stabilization devices 808 and 810 to rotate about a common center of rotation and to function as one unit.

Referring now to FIG. 8B, a simplified illustration of two dynamic stabilization devices 820 and 822 is provided to show relative movement. In this simplified illustration, the upper vertebra may be represented by block 824 and the lower vertebra may be represented by block 826. In actual practice, the blocks 824 and 826 would be coupled to the dynamic stabilization devices 820 and 822 via bone anchors (not shown). In this exemplary illustration, the dynamic stabilization devices 820 and 822 are similar to the dynamic stabilization device 200 (FIG. 2A) in that movement about the ends of the elbows may be restricted to an imaginary spherical surface. Although the dynamic stabilization devices 820 and 822 are illustrated in this manner, these devices are but examples. Any of the dynamic stabilization devices disclosed herein or any combination of devices may be used in a similar manner.

In the system illustrated in FIG. 8B, the dynamic stabilization device 820 is placed to the left of an imaginary sagittal plane and the dynamic stabilization device 822 is placed to the right of the imaginary sagittal plane such that each device points to the same center of rotation “A”.

With additional reference to FIGS. 8C-8F, the dynamic stabilization devices 820 and 822 of FIG. 8B are depicted in an approximately middle, neutral position (FIG. 8C), a flexion/extension position (FIG. 8D), a lateral bending position (FIG. 8E), and a rotation position (FIG. 8F). As shown in FIGS. 8C-8F, motion about all three axes may occur simultaneously, giving a combination of flexion/extension, lateral bending, and rotation. As depicted in FIG. 8B, the pivots of each of the joints of both elbows will point to the same center of rotation “A”.

In operation, each first and second dynamic stabilization devices 820, 822 move in conjunction with adjacent upper and lower vertebrae as the spine moves. For example, as a person bends forwards or backwards, the dynamic stabilization devices 820, 822 extend or retract as required, thereby allowing the anchors to move with the corresponding upper and lower vertebrae (represented by blocks 824 and 826) about one or more horizontal axes of rotation. As a person bends sideways right or left, the dynamic stabilization devices 820 and 822 bend to the right or left and extend or retract as required, depending upon which side of the spinous process the device is located, thereby allowing the first and second anchors to move with the corresponding upper and lower vertebrae. As a person rotates their torso to the left or to the right, the dynamic stabilization devices 820 and 822 skew to the right or left, adjusting themselves as required, thereby allowing the first and second anchors to move with the corresponding upper and lower vertebrae. As the dynamic stabilization devices 820 and 822 adjust in conjunction with the relative movement of adjacent vertebrae, the corresponding anchors to which braces are coupled can move with the corresponding adjacent upper and lower vertebrae, thereby maintaining the intended mechanical unloading or partial un-loading of forces upon an inter-vertebral disc while simultaneously allowing a full range of movement of the vertebrae.

Dynamic Systems and Devices that Permit Two Dimensional Movement:

As previously discussed, one of the purposes of the various embodiments of the disclosed dynamic stabilization devices is to enable adjacent pedicles the freedom to follow a curved motion which approximates their natural motion around a center of rotation as they move with respect to each other. In certain embodiments, some amount of translation is permitted such that the center of rotation need not be a fixed point. Furthermore, in some embodiments, there may be a need for planar movement. In other words, in some instances, it may be desirable to use a device which only allows two dimensional movement—as opposed to three dimensional movement.

The disclosed aspects of the embodiments could be modified to permit only two dimensional movement about a center of rotation. For instance, if the post portion 411 of brace 404 (described in reference to FIGS. 4A-4C) were to have a constant rectangular cross-section (i.e., a cross section that did not vary along the longitudinal axis), the brace 404 would only permit two dimensional movement (rotation about the X-axis).

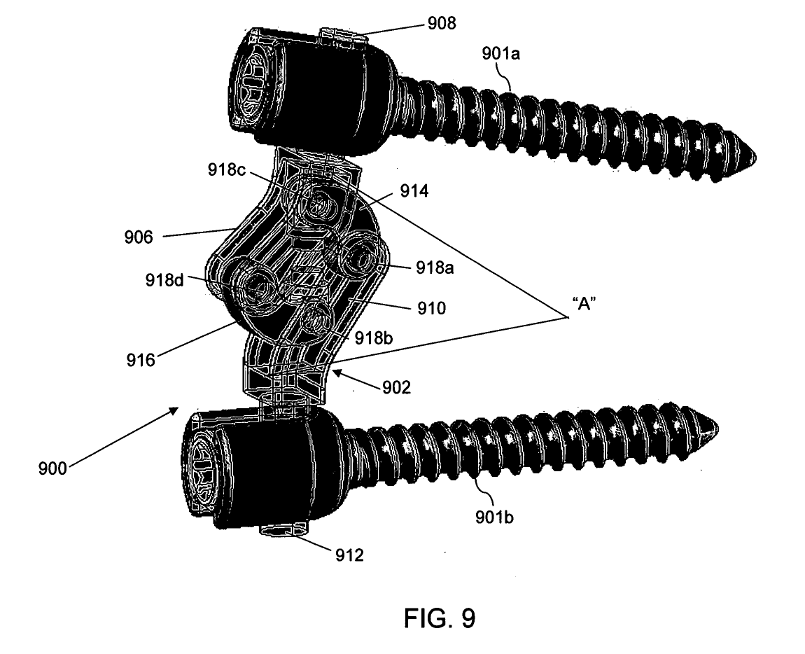

Similarly, if pins were used without rod end bearings in the four bar embodiments, only two dimensional movement would be possible. FIG. 9 describes such an embodiment.

FIG. 9 is an isometric drawing illustrating an embodiment of a four bar dynamic stabilization system 900 that is conceptually similar to the dynamic stabilization system 500 described with reference to FIG. 5A. In certain embodiments, the dynamic stabilization system 900 may create an anterior distracting force for providing substantially even unloading of inter-vertebral discs and allows limited movement about an imaginary two dimensional curve.

The dynamic stabilization system 900 comprises bone anchors 901a and 901b coupled to a dynamic stabilization device 902. The dynamic stabilization device 902 comprises an upper connecting member 906 coupled to an upper shank member 908, a lower connecting member 910 coupled to a lower shank member 912, and a first coupler member 914 and a second coupler member 916 interlinked with the upper and lower connecting members for movement. The dynamic stabilization device 902 may also include one or more spring members (not shown) for providing a force for controlling the movement between the upper connecting member 906 and the lower connecting member 910. In certain embodiments, such spring members may act to progressively break the movement or to provide a distracting mechanism or both. Each coupler member 914, 916 is rotatably connected at either end thereof to one of the connecting members 906, 910 to form a flexible, trapezoidal linkage.

In this embodiment, the upper connecting member 906 comprises a yoke portion. The lower connecting member 910 is similarly constructed. Each coupler member 914, 916 includes bores (not shown) that align with similar bores 918a-918d on the corresponding yoke portion of each of the connecting members 906 and 910. A pin member (not shown) joins and secures the upper and lower connecting members 906 and 910 to the coupler members 914 and 916 to enable a curvilinear rotation about a point “A.”

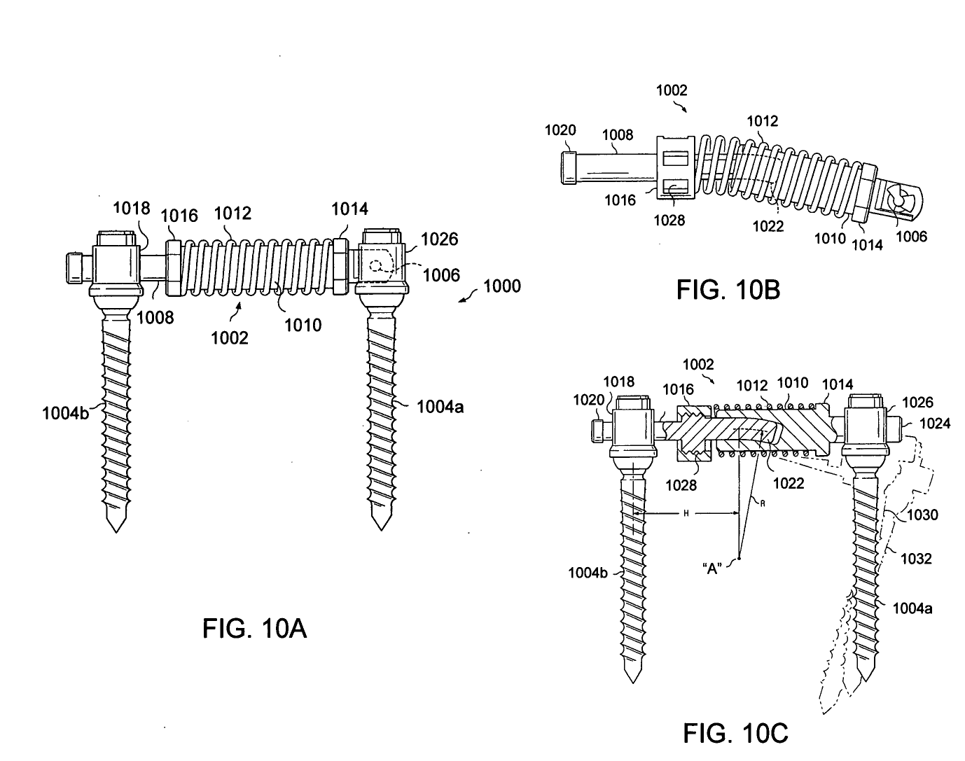

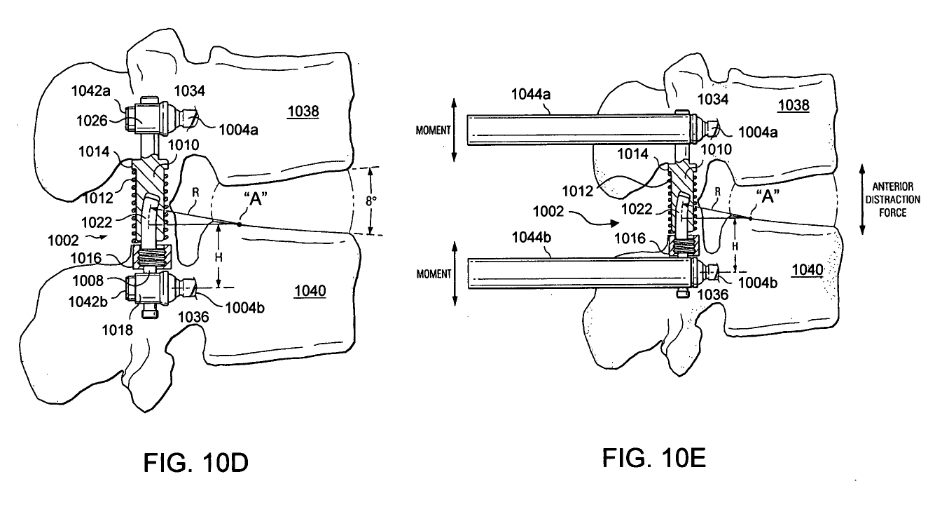

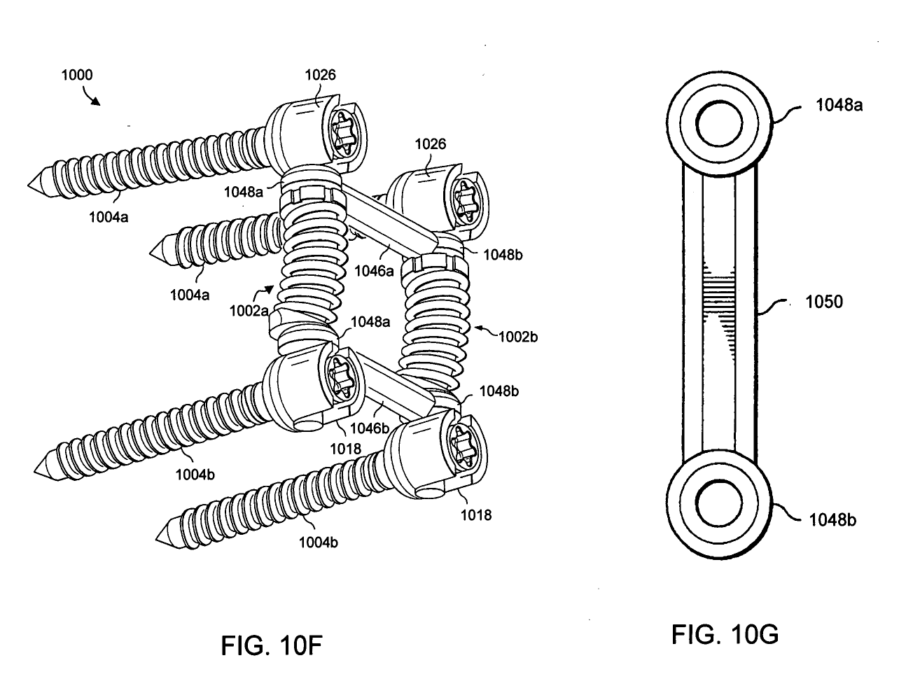

Other two dimensional embodiments and configurations are also possible. For instance, referring to FIG. 10A, there is illustrated an exemplary embodiment of a dynamic stabilization system 1000 for use between vertebrae (not shown). A dynamic stabilization device 1002 spans between two bone anchors (e.g., pedicle screws) 1004a and 1004b. The dynamic stabilization device 1002 includes brace portions 1008 and 1010 (which may be a tube within a tube) that are free to move with respect to each other along their longitude axis in a telescoping manner. Portion 1006 of brace portion 1010 may be attached to one pedicle screw while brace portion 1008 is attached to a second pedicle screw. Adjustment along the Y-axis may be achieved by moving the position of brace portion 1008 with respect to the pedicle screw prior to clamping the pedicle screw to dynamic stabilization device 1002. This effectively changes the neutral length of dynamic stabilization device 1002.

As stated above, the brace portions 1008 and 1010 may move with respect to each other along their longitude axis in a telescoping manner. This motion is controlled, in part, by one or more springs 1012. Stop 1014, working in conjunction with stop 1016, serves to allow spring 1012 to be effectively lengthened or shortened, thereby changing the force the spring exerts which, in turn, changes the force between brace portions 1008 and 1010. In the present example, the relative movement between brace portions 1008 and 1010 allows for approximately 5° to 20° flexion of the vertebrae to which the dynamic stabilization device 1002 is attached. Of course, the implementation of dynamic stabilization device 1002 may be adapted to allow for any desired range of flexion in alternative embodiments. In addition, as will be detailed, dynamic stabilization device 1002 may maintain a correct biomechanical center of rotation as it bends. The center of rotation is not necessarily limited to a fixed center of rotation with respect to the vertebrae. The dynamic stabilization device 1002 may also reduce or eliminate pressure on the disc between the vertebrae. This partial off-loading of the disc is accomplished by the rigid nature of the rod and spring assembly. If rotation of the dynamic stabilization device 1002 (e.g., rotation of the brace portion 1008 with respect to the brace portion 1010) becomes an issue, the telescoping portions can be designed, for example, using an interlocking groove or using matched longitudinal channels, one in each tube, to prevent relative rotation.

By changing the position where a head 1018 of pedicle screw 1004b grips portion 1008, the center of rotation in a superior/inferior axis of rotation along the patient's skeletal anatomy can be adjusted. Dynamic stabilization device 1002 can be adjusted to create a proper distraction height prior to being implanted and thereafter can be adjusted to the desired distraction force in situ. Because the spine is free (subject to constrained motion) to bend, multiple dynamic stabilization devices can be used along the spine while still allowing the spine to move into flexion and, if desired, extension. In certain procedures, the dynamic stabilization device 1002 may be, for example, positioned and correctly tensioned/adjusted in communication with a device that determines a patient's spinal neutral zone.

FIG. 10B shows the dynamic stabilization device 1002 extended when the spine is in flexion. In this scenario, the dynamic stabilization device 1002 extends around a curvilinear path and the spring length increases, in this example, from approximately 0.745 to 0.900 inches, with spring deflection of approximately 0.155 inches. End 1020 of brace portion 1008 is assumed in a fixed position while the portion 1006 moves superior (right) and exterior (down) with respect to the end 1020. Of course, other dimensions of increasing length and deflection may be achieved in other uses. That is, different amounts of flexion and extension may be permitted in certain patients.

FIG. 10C shows dynamic stabilization device 1002 in partial section attached to pedicle screws 1004a and 1004b. One end of portion 1008 is held captive by head 1018 positioned at the top of pedicle screw 1004b by a polyaxial connection. The portion 1010 of dynamic stabilization device 1002 slides over a curved post portion 1022 of portion 1008. In this embodiment, portion 1008 (and the post portion 1022) can be hollow or solid and portion 1010 will be at least partially hollow. An end 1024 of portion 1010 is held captive by head 1026 polyaxially mounted to pedicle screw 1004a. It is noted that end 1024 may be adjusted to extend beyond head 1026 prior to being clamped into head 1026 if it is necessary to allow for a greater range of travel of the post portion 1022 within tube 1010. For example, this may be necessary for closely placed bone anchors. As discussed, the spring 1012 may be positioned around the outside of portion 1010 between stops 1016 and 1014. In certain embodiments, the spring 1012 may be held in compression and adjusted by the rotatable stop 1016 moving under control of threads 1028.

As discussed, the post portion 1022 fits inside of portion 1010 and may be curved. It is this curve that allows pedicle screw 1004a to move in an arc (as shown) when the pedicle to which pedicle screw 1004a is attached rotates in flexion. This allows the dynamic stabilization system 1000 to rotate about center of rotation “A” with a curved motion which approximates the natural motion of the spine (where the term “natural” represents movement of a properly working spine). It is noted that the X-axis center of rotation of dynamic stabilization device 1002 is controlled by the bend of post portion 1022 relative to portion 1010. As discussed above, the center of rotation in the superior/inferior axis (Y-axis) is controlled by the position of end 1020 with respect to the pedicle screw 1004b.

Positions 1030 and 1032 (shown in dashed lines) of pedicle screw 1004a illustrate pedicle screw kinematic analysis as the spine moves into flexion. As shown, pedicle screw 1004a goes through a range of arc motion around center of rotation “A”. It is this range of arc motion that the dynamic stabilization device 1002 tries to maintain.

FIG. 10D shows dynamic stabilization device 1002 positioned in pedicles 1034 and 1036 of vertebrae 1038 and 1040, respectively. The length of the dynamic stabilization device 1002 between heads 1026 and 1018 may be adjusted during implantation until locking mechanisms 1042a, 1042b (in the heads 1026 and 1018, respectively) are tightened to secure the length of the dynamic stabilization device 1002 when the H dimension is as desired. This, as discussed, is the (Y) axis (or superior/inferior) of adjustment. The curvilinear motion may be set with respect to the R dimension and this is the (X) axis (or flexion/extension) of adjustment. The (X) and (Y) dimensions may be set with reference to the desired center of rotation “A”. The force provided by spring 1012 in combination with brace portions 1008 and 1010 keep vertebrae 1038 from pressing too heavily on the lower vertebra 1040, thereby partially off-loading stress from the intervertebral disc.

FIG. 10E shows that by applying a moment about extensions 1044a and 1044b and then locking down the length of dynamic stabilization device 1002 (e.g., to the dimension H) there can be created an anterior distraction force on vertebral bodies 1038 and 1040. This will more evenly distribute the loading on disc, thereby creating a more optimal environment for the disc when compared to only a posterior distracting implant system. Extensions 1044a-1044b are removed after the proper length of dynamic stabilization device 1002 is achieved.

FIG. 10F shows a dynamic stabilization system 1000 (FIG. 10A) interconnected with one or more cross-connectors 1046a and 1046b. The cross-connectors 1046a 1046b may be fixed or adjustable, and straight or curved as desired. The cross-connectors 1046a and 1046b may have various cross-sections, including a bar, plate, or tube as shown. Each cross-connector 1046a and 1046b acts to combine the dynamic stability provided by dynamic stabilization devices 1002a and 1002b into a single assembly and to provide a more fluid motion. As shown in FIG. 10G, each cross-connector 1046a and 1046b may be independent with a longitudinal member 1050 having openings 1048a and 1048b at its ends to receive brace members 1008, 1010 (FIG. 10A) of a dynamic stabilization devices 1002a and 1002b. Alternatively, one or both of the dynamic stabilization devices 1002a and 1002b and one or both of the cross-connectors 1046a and 1046b may be constructed as a single unit.

Spinous Process Embodiments:

Many of the embodiments disclosed herein are attached to the pedicles by means of pedicle anchors. However, such embodiments are not meant to limit the disclosed aspects. Those skilled in the art would recognize that many more embodiments are possible using the teachings of the disclosed invention.

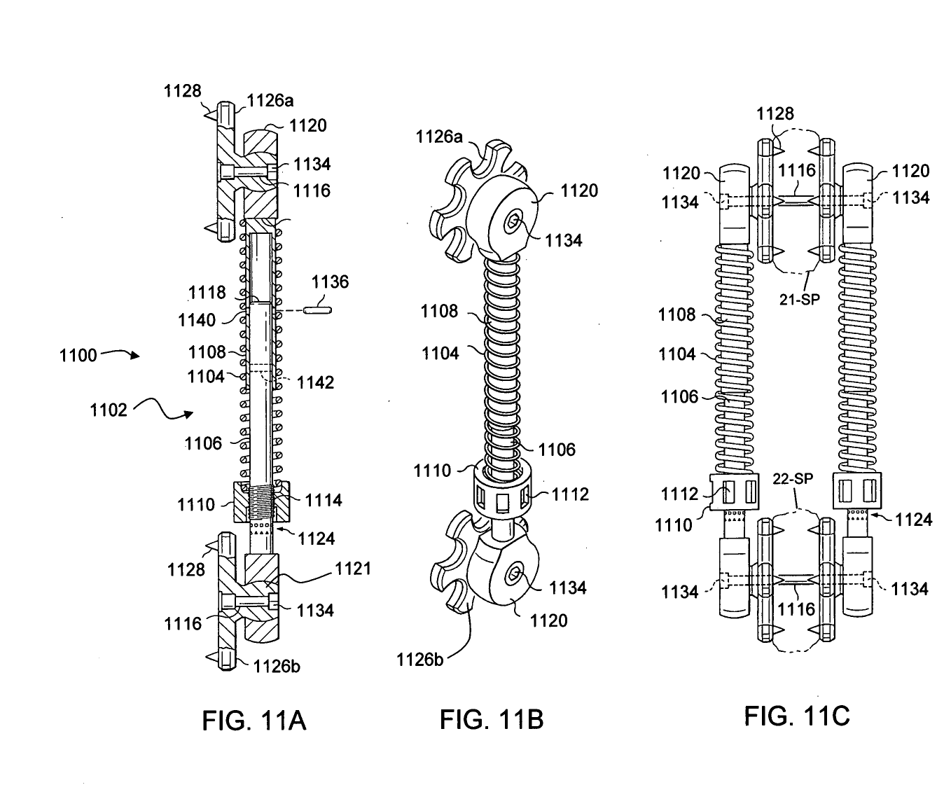

For instance, FIG. 11A shows a cross-section of one embodiment of a spinous process dynamic stabilization system 1100. The system includes a dynamic stabilization device 1102 having a brace comprising an external spring 1104 and a pair of expandable brace portions 1106 and 1108. Portion 1106, which can be a solid rod, if desired (or any other suitable structure, such as a tube, a plurality of parallel-arranged rods or tubes, etc.), moves inside portion 1108 which can be a hollow tube. External to both of these portions is the spring 1104, the tension of which is controlled by tightening (or loosening) stop 1110 under control of openings 1112 (FIG. 11B). Stop 1110 in this embodiment works in cooperation with threads 1114. Note that any type of stop can be used (e.g., threaded or threadless) and the stop(s) can be inside the rod or outside. Dynamic stabilization device (or “brace” or “rod”) 1102 can be attached to either side of the spinous process or could be used in pairs interconnected by rod 1116 (FIG. 11C).

As the spinous process moves into flexion, brace portion 1108 moves upward. Brace portion 1106 may remain relatively stationary and thus rod end 1118 may move down (relatively) inside portion 1108. This expansion and contraction along the lateral length of dynamic stabilization device 1102 allows the spine to follow a curved motion which approximates the normal physiologic motion during bending of the spine. Forward, lateral, and twisting motions of dynamic stabilization device 1102 may be accomplished by a rod end 1120 that is free to move in three planes or axes around spherical end bearing 1121.

Stop 1110 may be moved to adjust the tension of spring 1104. In the present example, force increases as stop 1110 is moved upward and force decreases as the stop is moved downward. Force marks (e.g., triangles and squares 1124 shown in this example) embossed (or otherwise marked) on shaft 1106 aid the surgeon in adjustment of the spring force. Thus, for instance, the triangles may indicate that positioning the stop at their location results in a spring force of, for example, thirty pounds, while the squares may indicate that positioning the stop at their location results in a spring force of, for example, sixty pounds. This pre-calibration may help the installation process. It is noted that the spacing between the force marks in the drawings are arbitrarily drawn in this example, but may be implemented so as to represent the difference between forces.

Load transfer plates 1126a, 1126b may help distribute the forces between the respective vertebrae. Spikes 1128 may be used for better load distribution to the spinous process.

FIG. 11B shows dynamic stabilization system 1100 from a perspective view. The rod ends 1120 of dynamic stabilization device 1102 revolve around rod end bearings 1121 and allow rotation of the device for flexion/extension, lateral bending, and trunk rotation. Fastener 1134 serves to hold the dynamic stabilization device 1102 to the end support.

FIG. 11C shows one embodiment of a pair of dynamic stabilization devices 1102 connected on either side of spinous process 21-SP (22-SP). Each dynamic stabilization device 1102 may be installed by creating a hole (by drilling or other means) in each spinous process and screwing (or otherwise connecting) rod 1116 through the created hole to interconnect the two internally separated devices, as shown.

Cover:

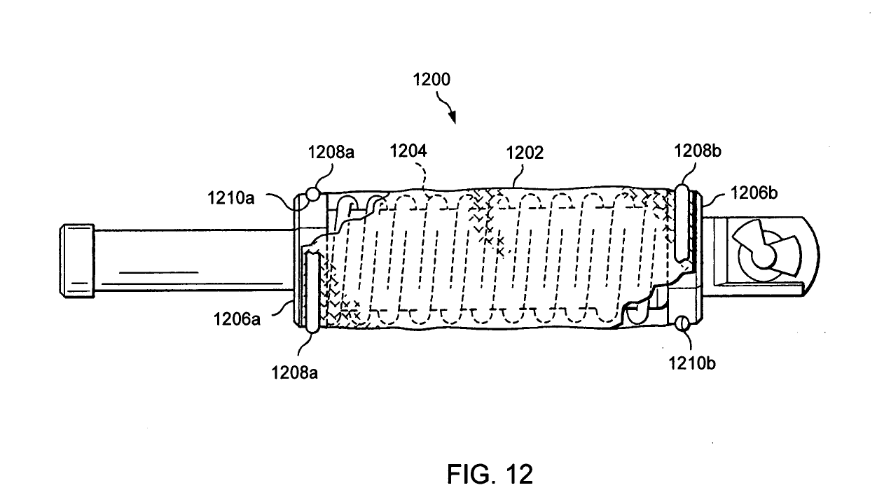

FIG. 12 shows alternative embodiment of a dynamic stabilization brace 1200 having cover 1202 surrounding a spring 1204. In this embodiment, the ends of cover 1202 are held to stops 1206a and 1206b by rings 1208a and 1208b. The rings 1208a and 1208b may be fitted into slots 1210a and 1210b, respectively. The cover may used to protect the device from being interfered with once implanted. In certain embodiments, the cover (or sleeve) 1202 can be constructed from an elastomeric material, a surgical fabric, and/or polyester, as examples. It is contemplated that any of the embodiments described herein may be used with a cover similar to cover 1202 or an equivalent elastomeric cover. Such an elastomeric cover may also provide a progressive breaking action.

Locking Feature:

Note that in any of the embodiments shown, the spring force can be increased to a point where the device effectively becomes static in order to achieve fusion. Also, in the embodiments using telescoping members, one or more holes could be positioned through the slide portions such that when a pin is inserted through the holes, the pin effectively prevents the brace from further expansion or contraction. For example, with reference to FIG. 11A, a pin 1136 may be inserted through holes 1140 and 1142 in portions 1108 and 1106, respectively. The pin could, for example, have spring loaded balls (or any other mechanism) that serve to prevent the pin from easily pulling out of device 1100 once inserted. In addition, the spacing stop 1110 could be tightened, either permanently or on a temporary basis, to a point where spring tension effectively places the device in a static condition in order to promote fusion of the treated vertebrae in situations where motion preservation fails to meet surgical end-goals.

In embodiments where linkages are used, a pinned or hinged mechanism may be replaced with a screw system that would effectively lock the linkage in place. Alternatively, other methods may be used to lock an existing pin or hinge mechanism.

Neutral Zone Discussion:

It is noted that with certain embodiments of the present invention, it is possible to take neutral zone displacement readings so as to be able to tension a dynamic stabilization system properly with respect to a patient. Based on the readings, the X, Y, and Z axes can be adjusted. A dynamic stabilization system may be sensitive to proper placement of the device to restore proper kinematics and range of motion, and avoid causal deleterious effects of increasing rate of degeneration on adjacent segments. A neutral zone device is a device that can aid in the placement of the dynamic stabilization device by determining the center of rotation in flexion/extension. Once this center of rotation has been determined, the device can be located to best reproduce that center of rotation. The neutral zone device will cycle the spine through a range of motion measuring forces throughout the range of motion. Also, the device can be used after device implantation to confirm proper implant placement.

The embodiments discussed herein reproduce the natural motion of the spine while immobile. As shown herein, the embodiments create a curved two or three dimensional path for relative movement between the pedicles which creates, restores and controls the normal center of rotation. Other embodiments that would produce the proper motion could include, for example:
a) a guide bar comprising a pair of pins articulating in a matching pair of slots where the slots would diverge to produce a curvilinear motion of a point on the guide bar;
b) any type of curvilinear guides made up of male and female shapes following a curved path with a geometric cross section (e.g., dovetail, T-slot, round, square, rectangle) cross-sectional geometry; and/or
c) a four or five bar mechanism that would produce a curved path of the pedicle screw.

Having thus described aspects of the present invention by reference to various embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered obvious and desirable by those skilled in the art based upon a review of the foregoing description of preferred embodiments.

What is claimed is:

1. A spine stabilization device, comprising:
a brace adapted to span between a first bone anchor and a second bone anchor, the brace including:
a first joint; and
a second joint; wherein the brace allows for movement between the first joint and the second joint such that the movement of the second joint with respect to the first joint is generally restricted to vertical and horizontal movement along a three dimensional curved path surface having a substantially constant radius about a center of rotation wherein the center of rotation is positioned outside of the brace.

2. The spine stabilization device of claim 1, further comprising a distracting mechanism coupled to the first joint and the second joint to exert a force between the first joint and second joint.

3. The spine stabilization device of claim 1, wherein the center of rotation is substantially positioned within a spine disc space when the device is implanted between two vertebrae.

4. The spine stabilization device of claim 1, wherein the brace further comprises:
a third joint;
a first link coupled to the first joint and the third joint; and
a second link coupled to the second joint and the third joint.

5. The spine stabilization device of claim 4, wherein movement of the third joint is generally restricted to a generally curved path having the constant radius about the center of rotation.

6. The spine stabilization device of claim 5, wherein the first, second, and third joints are pin joints.

7. The spine stabilization device of claim 6, wherein each pin joint has a pin having a longitudinal axis which intersects the center of rotation.

8. The spine stabilization device of claim 1 wherein the first joint is coupled to a first member and the second joint is coupled to a second member.

9. The spine stabilization device of claim 8 further comprising a means for creating a force between the first member and the second member.

10. The spine stabilization device of claim 8 further comprising an exterior cover positioned around the first and second links members.

11. A spine stabilization system comprising:
a first bone anchor;
a second bone anchor;
a brace spanning between the first bone anchor and the second bone anchor, the brace including:
a first member coupled to the first bone anchor;
a second member coupled to the second bone anchor, wherein the first member and the second member are slideably mated along a portion of their longitudinal lengths such that the movement of the second member with respect to the first member is generally restricted to vertical and horizontal movement along a three dimensional curved path surface having a substantially constant radius about a center of rotation, wherein the center of rotation is positioned outside of the brace.

12. The spine stabilization system of claim 11 wherein the first and second bone anchors are anchors adapted to attach to a spinous process of a vertebra.

13. The spine stabilization system of claim 11 further comprising a three-axis rotational bearing connection for coupling the first member to the first bone anchor and the second member to the second bone anchor.

14. The spine stabilization system of claim 11 wherein the brace further comprises a means for creating a force between the first member and the second member.

15. The spine stabilization system of claim 14 further comprising a means for adjusting the force between the first member and the second member.

16. The spine stabilization system of claim 11 further comprising a cover positioned partially around the first and second members.

17. The spine stabilization system of claim 11 further comprising a means to positionally lock the first member relative to the second member.

18. A method for spine stabilization comprising:
inserting a first bone anchor into a first vertebra;
inserting a second bone anchor into a second vertebra;
attaching a first joint to the first bone anchor;
attaching a second joint to the second bone anchor; and
interconnecting the first joint and the second joint to create a brace that spans the first bone anchor and the second bone anchor, such that the first joint and the second joint are slideably mated along a portion of their longitudinal lengths;
wherein the brace allows for movement between the first joint and the second joint such that the movement of the first joint with respect to the second joint is generally restricted to vertical and horizontal movement along a three dimensional curved path surface having a substantially constant radius about a center of rotation, wherein the center of rotation is positioned outside of the brace.

19. The method of claim 18 wherein the step of interconnecting the first joint and the second joint further comprises:
interconnecting a third joint to the first joint with a first link; and
interconnecting the third joint to the second joint with a second link;
wherein the third joint is generally restricted to a generally curved path having the constant radius about the center of rotation.

20. The method of claim 19 wherein the first joint, the second joint and the third joint are pin joints.
